(19) United States
(12) Patent Application Publication
(10) Pub. No.: US 2012/0121574 A1
(43) Pub. Date: May 17, 2012

(54) ANTIMICROBIAL, ANTIVIRAL, ANTICANCER AND IMMUNOMODULATORY PEPTIDES AND USES THEREFORE

(76) Inventor: Luciano Polonelli, Parma (IT)

(21) Appl. No.: 13/101,122

(22) Filed: May 4, 2011

(30) Foreign Application Priority Data
Nov. 15, 2010 (US) PCT/US2010/056763

(51) Int. Cl. A61K 39/395; C07K 5/117; C07K 5/083; A61P 35/00; A61P 31/04; A61P 31/10; A61P 31/12; A61P 37/02; C07K 7/06; C07K 5/113

(52) U.S. Cl. 424/130.1; 530/328; 530/327; 530/330; 530/329

(57) ABSTRACT

Polypeptides derived from constant domains of antibody light (L) and/or heavy (H) chains as well as from complementary determining regions (CDRs) of immunoglobulin variable regions are disclosed possessing broad spectrum biological activities including, among others, antifungal, antibacterial, antiviral, anticancer and/or immunomodulatory activity in vitro, ex vivo and/or in vivo.

### RELATED APPLICATIONS

This invention is filed as a Continuation-in-Part and claims the benefit of PCT/US10/56763 filed Nov. 15, 2010, and Provisional application Ser. No. 61/261,738 filed Nov. 16, 2009.

### SEQUENCE LISTING

The instant application contains a Sequence Listing which has been submitted in ASCII format via EFS-Web and is hereby incorporated by reference in its entirety. Said ASCII copy, created on May 4, 2011, is named RAS1001C.txt and is 10,421 bytes in size.

### FIELD OF THE INVENTION

This invention relates to the field of immunology, particularly to peptides derived from antibody light (L) and heavy (H) chain amino acid sequences, and derivatives thereof, that are active in immunologically related processes. More specifically, this invention relates to the use of such peptides possessing antibacterial, antifungal, antiviral, antitumor and/or immunomodulatory activities in hosts to which said peptides are introduced, for treating fungal, viral, and bacterial infections and immunologic and cancerous disorders in mammals including humans, farm and other domesticated and/or zoo-kept animals.

### BACKGROUND OF THE INVENTION

The following information includes subject matter that may be useful in understanding the present invention. It is not an admission that any such information is prior art, or relevant, to the presently claimed inventions, or that any publication specifically or implicitly referenced is prior art.

Immunoglobulins (Igs) otherwise commonly referred to as antibodies (Abs) are composed of two identical sized L chains (23 kD) and two identical H chains which range in size, (i.e., between 50-70 kD). The chains are connected by inter-chain disulfide bonds; the H and L chains and the two H chains are held together by inter-chain disulfide bonds and by non-covalent interactions. The number of inter-chain disulfide bonds varies among different Ig molecules. There are also intra-chain disulfide bonds within each of the polypeptide chains. FIG. 1 depicts, schematically, generally accepted aspects of Ab L and H chain construction. As can be understood by one of skill in the art, each of the H and L chains comprise a “constant” (C) region and a “variable” (V) region. The C region of the H chains makes up the greater portion of the amino acid sequence making up the Ab. The H chain C region is broken up into recognized domains labeled CH1-4. L chains only have one C region domain labeled CL. Igs also have a “hinge” region. This is the region at which the arms of the Ab molecule form a Y. It is called the hinge region because there is some flexibility in the molecule at this region.

When the amino acid sequences of many different H chains and L chains were compared, it became clear that both the H and L chain could be divided into two regions based on variability in the amino acid sequences and each L and H chain comprised a V and a C region, as stated above, i.e., 1) L chain—V_{L} (110 amino acids) and C_{L} (110 amino acids), 2) H chain—V_{H} (110 amino acids) and C_{H} (330-440 amino acids). Igs are further structured in that they are folded into globular regions each of which contains an intra-chain disulfide bond (FIG. 1). These regions are the earlier referenced domains (FIG. 1). Specifically, L chain domain, i.e., V_{L} and C_{L}, and the H chain domains—V_{H}, C_{H1}-C_{H3} (or C_{H4}). Additionally, carbohydrates in the form of oligosaccharides are attached to the C_{H2} domain in most Igs. However, in some cases carbohydrates may also be attached at other locations.

Comparisons of the amino acid sequences of the V regions of Igs show that most of the variability resides in three regions called the hypervariable regions (HVRs) or the complementarity determining regions (CDRs). CDRs are found in both V_{H} and V_{L}. Finally, the regions between the CDRs in the V region are called the framework regions.

Various amino acid sequence segments of Ab V sequences of L and H chains, specifically CDRs or CDR-related peptides, can possess antimicrobial (i.e., antibacterial and/or antifungal, whereby by antifungal is meant antiyeast and/or antimold), antiviral, anticancer and/or immunomodulatory activities that may be beneficial against certain fungal, bacterial and viral infections and immunological or cancer disorders (Polonelli et al., 2003, Cenci et al., 2006, Polonelli et al., 2008, Gabrielli et al., 2009). The fact that amino acid sequences comprising less than the whole Ab, normally consisting of a complete H and L chains, can possess antimicrobial, antiviral anticancer and/or immunomodulatory activities is surprising and suggests that beyond the half life of a typical Ig, fragments of the whole Ig molecule may have biological actions such as effectively influence the antiinfective and anticancer cellular immune response in a way reminiscent of regulatory peptides of innate immunity.

The finding that subsets of amino acid sequences within the V regions of the L and H chains have anti-infective activity, though nonetheless surprising, is comprehendible when considering the multiplicity of variation in amino acid sequence capable within the V region. A partly digested Ab could comprise just such a region that otherwise was specific for a family of microorganisms. However, it is not intuitive that the C region amino acid segments could harbor such activities as it is the conventional wisdom that the C regions merely play a structural role in presenting the active site (i.e., V region) to the antigen as well as provide attachment to cell membranes and effectors such as complement and receptors on immune effector cells termed Fc receptors. Given the ongoing need to advance the medical sciences, we disclose herein a new class of polypeptide within the CDR-derived peptide family that exhibits immune modulating activities. We further disclose the surprising and novel finding of a class of polypeptide derived from, exclusively, C region sections of Abs, whether L or H chain, exhibiting any of antibacterial, antifungal, (antiyeast and antimold), antiviral, anticancer and immunomodulatory activities against a wide spectrum of microbial agents and tumor and immunological disorders.

Additionally, it is well established that many invasive bacterial, and fungal and viral infections are difficult to treat. To date there have been some advancement in the development of antibacterial, antifungal, and antiviral agents for use in treatment regimens.

Yet, over the last 3 decades, there has been a rise in the prevalence of opportunistic fungal infections concomitantly with an increase in the frequency of solid-organ and hematopoietic stem cell transplants (HSCTs), more aggressive chemotherapy, the AIDS epidemic, and advances in critical care. While Candida spp. and Aspergillus spp. remain the most common causes of invasive fungal infections (IFIs) in immune-compromised hosts, infections due to other fungi are seen with increased frequency (Arendrup, 2009; Erjavec et al., 2009; Zilberberg and Shorr, 2009). IFIs have historically been associated with high morbidity and mortality, partly because of the limitations of available antifungal therapies and difficulties in making a rapid and accurate diagnosis (Sable et al., 2008). In addition to being a growing clinical challenge, drug resistant and multidrug-resistant human pathogenic fungi are also neglected potential bioterrorism agents (Casadevall and Pirofski, 2006). In particular, human pathogenic fungi are easily obtainable from the environment, highly dispersible and can cause significant disease after inhalation with relatively low inocula (Casadevall and Pirofski, 2006).

Since the late 1950s, the standard of care for treatment of serious fungal infections had been amphotericin B, an intravenous (IV)-only agent with significant toxicity (Kauffman and Carver, 2008; Cornely et al., 2009; Moen et al., 2009; Rogers and Frost, 2009). The 1990s saw the introduction of lipid formulations of amphotericin B, as well as the triazoles fluconazole and itraconazole (Fera et al., 2009; Moen et al., 2009; Rogers and Frost, 2009). Although these agents offered clear advantages over amphotericin B, they were limited by formulation, spectrum of activity, and/or the development of resistance. In the past decade, there have been major advances in therapy. Broader-spectrum triazoles (voriconazole and posaconazole) and the new echinocandin class of antifungals (caspofungin, micafungin, and anidulafungin) have been introduced in the current decade, and noninvasive diagnostic methods have improved (Fera et al., 2009; Gergis et al., 2009; Rogers and Frost, 2009).

Triazole antifungal drugs, such as fluconazole, voriconazole, itraconazole, and posaconazole, owe their success to better clinical safety profiles than the single effective fungicidal agent, amphotericin B, that was available before their introduction. However, amphotericin B formulations are still employed for clinically challenging infections like deep candidal infections. Resistance to azoles is emerging in species that were previously susceptible, in particular Candida albicans. Echinocandins competitively inhibit the synthesis of an essential cell wall component, the 1,3-β-glucan, of Candida spp. and Aspergillus spp., but are generally inactive against other fungi, such as Cryptococcus neoformans (Rogers and Frost, 2009). All agents of this class are of parenteral formulations, with no oral preparations available. Reduced sensitivity to echinocandins, although uncommon, can result from mutations or overexpression of their target, 1,3-β-glucan synthases (Cappelletty and Eiselstein-McKitrick, 2007; Kauffman and Carver, 2008; Walker et al., 2008).

Although several antifungals have been licensed in the last 5 years, some patients remain difficult to treat. In particular IFI in immunocompromised patients, such as HSCT recipients and patients with acute leukemia during periods of profound neutropenia, are an increasingly common cause of mortality (Arendrup, 2009; Erjavec et al., 2009; Zilberberg and Shorr, 2009).

Thus, there is a need for new antifungal agents with a broad spectrum of activity, limited resistance potential, favorable safety profile and limited drug interactions. The main reasons for this need include intrinsic or acquired antifungal resistance, toxicity of existing agents with broad spectrum of activity, limited spectrum of activity of some of the safest available compounds, organ dysfunction preventing the use of some existing agents, and drug interactions of existing agents. To address this unmet clinical need, the present invention provides a heretofore untold novel class of polypeptides derived from both CDR and C regions of Igs, said peptides possessing a variety of activities including antifungal (antiyeast and antimold), antibacterial, antiviral, anticancer as well as immunomodulatory by direct action of the peptides, all the above of which activities are directly amenable to their respective effective use in treatment regimens.

### SUMMARY OF THE INVENTION

In a first embodiment, the invention comprises peptides derived from either the L or H chains of Igs. i.e., Abs. In a particularly preferred embodiment, invention peptides are derived from the C regions of the L and H chains of any class of Igs. In this aspect, C regions are meant to include C_{H} regions 1, 2, 3, and 4, the C_{L} region and the hinges, as such regions are well understood to those of ordinary skill in the arts. The discovery by the present inventors that peptide segments of the C regions possess antibacterial, antifungal (antiyeast and antimold), antiviral, anticancer and or immunomodulatory activities is novel as prior understanding of the skilled artisan is that only the V regions of L and H chains, and in particular the CDR-related segments thereof, possessed such activities.

In a second embodiment, peptides of the present invention possess a broad spectrum of activity, whether CDR-related or strictly non-CDR C region-derived amino acid sequence, comprising any or all of antimicrobial (antibacterial and antifungal) antiviral, anticancer and immunomodulatory activity, regardless of their origin in either C_{L} or C_{H} or, with respect to CDR-related peptides, V_{L} or V_{H} regions, exclusive of specific known amino acid sequences derived from any of said origins with known antimicrobial, antiviral or anticancer activity.

In a third embodiment, individual peptides of the invention exhibit alone or in combination any of antimicrobial (antibacterial and antifungal), antiviral, anticancer and immunomodulatory activity in vivo in a host mammal. In a related embodiment, the antimicrobial (antibacterial and antifungal), antiviral, anticancer and immunomodulatory activity of the peptides is unrelated to any activity of the complete Ig from which they are derived.

In another embodiment, the peptides can be naturally occurring peptides derived from L or H chains of Igs or synthetic derivatives thereof wherein one or more amino acids of the peptide sequences are substituted with other amino acids such as alanine, for example. In a related embodiment, the peptides of the invention are generally between 4 and 20 amino acids in length, more usually between 4 and 16 amino acids in length, and even more typically between 4 and 12 amino acids in length. Most commonly the peptides are between 4 and 10 or 4 and 11 amino acids in length. Further, the amino acid sequences of the bioactive antibody peptides possess a primary family structural motif that as one of ordinary skill in the art will recognize comprises amino acids of one type (e.g., polar, nonpolar, charged, noncharged, bulky versus small R group, etc.) interchanged with amino acids of another type. Specifically, with respect to the invention polypeptide family comprising broad spectrum antimicrobial, anticancer, immunomodulatory, and other activities, the structural unity is found in the general formula XZ*1X1Z*11XZ (consensus sequence 1) as described in detail below. Additionally, the peptides can be described as possessing further unity through their respective relationships found in alternative structural formulas as also disclosed herein (consensus sequences 2, 3, 4, 5 and 6). With respect to said formulas, amino acid substitutions are allowable as one of skill will recognize the evolutionarily acceptable amino acid substitutions commonly found in peptide families possessing similar activities. Further still, the peptides may also possess a beta sheet secondary structure. In additional embodiments, the naturally occurring Ig C region and CDR region peptides, as well as their alanine, serine, or otherwise 80% and 90% sequence identity with the native isolated sequence possess a similar primary structure given the evolutionarily accepted equivalent amino acids substitutions as depicted in Table IV. Thus, for every 10 amino acids in such a C region derived polypeptide, one or two amino acids can likely be substituted for an amino acid not naturally found in that sequence location while preserving considerable activity or even improving the activity of said peptide's antimicrobial, antiviral immunomodulatory or anticancer activities and remaining within a base level structural unity based on similar charge configurations through the consensus formulas. In a related embodiment, peptides of the invention derived from the C domain, identified by Seq. Id. Nos. 14-24 possess 90% sequence identity with one another and with Seq. Id. No. 1, while Seq. Id. No. 25 possesses an 80% sequence identity with Seq. Id. No. 1. Peptides of the invention derived from the C domain, identified by Seq Id. Nos. 27-37 possess 90% sequence identity with one another and with Seq. Id. No. 2. Additionally, peptides of the invention derived from the CDR domain identified by Seq. Id. Nos. 5-13 possess 90% sequence identity with one another and with Seq. Id. No 4, and further, all of these (i.e., Seq. Id. Nos. 4-13) possess a similar activity against a target, here immune cells.

In another embodiment, the peptide having antimicrobial (antibacterial and antifungal), antiviral, anticancer, and/or immunomodulatory activity can be used in therapeutic regimens by topical and/or systemic administration. In such administration, topical application can comprise cream and ointment bases including solvents, salts and absorbents as are well known in the arts. Other active and inert reagents can be included as appropriate for the healing arts.

Further still, in yet another embodiment, certain peptides of the disclosure are CDR-derived peptides that induce a protective anticandidal cellular immune response exclusively through an immunomodulatory activity despite their not possessing any direct candidacidal activity themselves. In a particularly preferred embodiment, the immunomodulatory activity was demonstrated by activities of synthetic peptides derived from the CDRs of a mouse monoclonal Ab (MoA) specific for the difucosyl human blood group A (Gabrielli et al., 2009).

### BRIEF DESCRIPTION OF THE DRAWINGS

The file of this patent contains at least one drawing executed in color. Copies of this patent with color drawings are provided to the Patent and Trademark Office with payment of the necessary fee.

FIG. 1 shows the Ab domain structure as is well known in the arts, namely, the two identical H chains comprising 3 to 4 C region domains (CH1-4), a V region (VH), itself comprising CDRs, and a hinge region as appropriately labeled. Also, the Ab has L chains comprising a C region (CL) and a V region (VL).

FIGS. 2A, B, and C show three basic classes of Ig molecules, namely IgG (FIG. 2A), IgM (FIG. 2B) and IgA (FIG. 2C), each represented in the form of a linear amino acid numbered bar chart, the V end region of the Ig amino acid sequence to the left and C region to the right. Each chart further indicates sections of C region super families making up parts of the C region domains. Each class of Ig, IgG, IgM, or IgA, is disclosed to have peptides, with antimicrobial, antiviral, anticancer and immunomodulatory activities, of the invention, each such peptide, N10K, T11F, and H4L, for example, as further described below, located in one or another portion of the Ig's C region.

FIG. 3 shows the in vitro activity of N10K against C. albicans SC5314 strain. Amount of N10K is in micrograms/ml.

FIGS. 4A and B show the in vivo activity of N10K against systemic candidiasis. In FIG. 4A the survival rate of subject mice is depicted. In FIG. 4B the yeast recovery rate per kidney is graphed.

FIG. 5 shows the in vitro activity of N10K against caspofungin resistant C. albicans strain UM4. Amount of N10K is in micrograms/ml.

FIG. 6 shows the in vitro activity of N10K against a caspofungin-resistant Saccharomyces cerevisiae strain YGR032W. Amount of N10K is in micrograms/ml.

FIG. 7 shows the in vitro activity of N10K against C. neoformans 6995 strain. Amount of N10K is in micrograms/ml.

FIG. 8 shows the in vitro activity of N10K against Malassezia furfur 101 strain. Amount of N10K is in micrograms/ml.

FIG. 9 shows the in vitro activity of N10K against Aspergillus fumigatus 1163 strain. Amount of N10K is in micrograms/ml.

FIG. 10 shows the in vitro activity of N10K against Staphylococcus aureus 29213 strain. Amount of N10K is in micrograms/ml.

FIG. 11 shows the in vitro activity of N10K against Escherichia coli ATCC 25922 strain. Amount of N10K is in micrograms/ml.

FIG. 12 shows the in vitro activity of N10K against Klebsiella pneumoniae ATCC 700603 strain. Amount of N10K is in micrograms/ml.

FIG. 13 shows the in vitro activity of N10K against Pseudomonas aeruginosa ATCC 25853 strain. Amount of N10K is in micrograms/ml.

FIGS. 14A and B show the in vitro activity of N10K against HIV IIIB (X4). In FIG. 14A the antiviral activity is depicted wherein the peptide concentration was 10 micrograms/ml administered every 4 days post viral infection. FIG. 14B is a graph showing that where cells were preincubated with N10K at the concentration of 10 micrograms/ml, a lesser antiviral activity is still present. Y axis shows concentration of HIV p24 (pg/ml).

FIG. 15 shows the in vitro activity of N10K against HIV BaL (R5). The antiviral activity is depicted wherein the peptide concentration was 10 micrograms/ml administered every 4 days post viral infection. Y axis shows concentration of HIV p24 (pg/ml).

FIG. 16 is a graph showing the in vitro activity of N10K against B16F10-Nex2 melanoma cells. Specifically, as the concentration of N10K increases (mM), the viability of the cancer cells decreases.

FIGS. 17A and 17B are graphs showing immunomodulatory activity of N10K. FIG. 17A shows induction by N10K of IL-6 by human monocytes. FIG. 17B shows the expression of Dectin-1 by monocytes stimulated by N10K or heat inactivated C. albicans (h.i. CA-6). NC is negative control, NS is absence stimulation. N10K at 10 micrograms/ml.

FIGS. 18A, B, and C show the phagocytosis of non opsonised C. albicans CA-6. FIG. 18A is a graph of % of phagocytic cells. FIG. 18B shows FACS indicating the medium number of yeast particles adhered or ingested by each monocyte. FIG. 18C is a Table showing the mean number of attached and ingested C. albicans.

FIGS. 19A and B show the production of cytokines IL-12p40 (FIG. 19A) and IL-6 (FIG. 19B) in human PBMCs stimulated by N10K (10 micrograms/ml).

FIG. 20 shows the in vitro activity of T11F against C. albicans SC5314 strain. Amount of T11F is in micrograms/ml.

FIG. 21 shows the in vitro activity of T11F against caspofungin resistant C. albicans strain UM4. Amount of T11F is in micrograms/ml.

FIG. 22 shows the in vitro activity of T11F against a caspofungin-resistant S. cerevisiae strain YGR032W. Amount of T11F is in micrograms/ml.

FIG. 23 shows the in vitro activity of T11F against C. neoformans 6995 strain. Amount of T11F is in micrograms/ml.

FIG. 24 shows the in vitro activity of T11F against M. furfur 101 strain. Amount of T11F is in micrograms/ml.

FIG. 25 shows the in vitro activity of T11F against A. fumigatus 1163 strain. Amount of T11F is in micrograms/ml.

FIG. 26 shows the in vitro activity of T11F against S. aureus 29213 strain. Amount of T11F is in micrograms/ml.

FIG. 27 shows the in vitro activity of T11F against E. coli ATCC 25922 strain. Amount of T11F is in micrograms/ml.

FIG. 28 shows the in vitro activity of T11F against K. pneumoniae ATCC 700603 strain. Amount of T11F is in micrograms/ml.

FIG. 29 shows the in vitro activity of T11F against P. aeruginosa ATCC 25853 strain. Amount of T11F is in micrograms/ml.

FIG. 30 shows the in vitro activity of T11F against HIV BaL (R5). The antiviral activity is depicted wherein the peptide concentration was 10 micrograms/ml administered every 4 days post viral infection. Y axis shows concentration of HIV p24 (pg/ml).

FIG. 31 shows the in vitro activity of T11F against B16F10-Nex2 melanoma cells. (mM concentration directly associated with decrease in cancer cell viability).

FIG. 32 shows the in vitro activity of H4L against C. albicans SC5314 strain. Amount of H4L is in micrograms/ml.

FIG. 33 shows the in vitro activity of H4L against C. neoformans 6995 strain. Amount of H4L is in micrograms/ml.

FIG. 34 shows the in vitro activity of H4L against M. furfur 101 strain. Amount of H4L is in micrograms/ml.

FIG. 35 shows the in vitro activity of H4L against A. fumigatus 1163 strain. Amount of H4L is in micrograms/ml.

FIG. 36 shows the in vitro activity of H4L against S. aureus 29213 strain. Amount of H4L is in micrograms/ml.

FIG. 37 shows the in vitro activity of H4L against E. coli ATCC 25922 strain. Amount of H4L is in micrograms/ml.

FIG. 38 shows the in vitro activity of H4L against K. pneumoniae ATCC 700603 strain. Amount of H4L is in micrograms/ml.

FIG. 39 shows the in vitro activity of H4L against P. aeruginosa ATCC 25853 strain. Amount of H4L is in micrograms/ml.

FIG. 40 shows the in vitro activity of H4L against HIV BaL (R5). The antiviral activity is depicted wherein the peptide concentration was 10 micrograms/ml administered every 4 days post viral infection.

FIG. 41 shows the in vitro activity of MoA V_{H}CDRH₃ against M. furfur 101 strain. Amount of MoA V_{H}CDR₃ is in micrograms/ml.

FIG. 42 shows the in vitro activity of MoA V_{H}CDRH₃ against A. fumigatus 1163 strain. Amount of MoA V_{H}CDR₃ is in micrograms/ml.

FIG. 43 shows the in vitro activity of MoA V_{H}CDRH₃ against S. aureus 29213 strain. Amount of MoA V_{H}CDR₃ is in micrograms/ml.

FIG. 44 shows the in vitro activity of MoA V_{H}CDRH₃ against E. coli ATCC 25922 strain. Amount of MoA V_{H}CDR₃ is in micrograms/ml.

FIG. 45 shows the in vitro activity of MoA V_{H}CDRH₃ against K. pneumoniae ATCC 700603 strain. Amount of MoA V_{H}CDR₃ is in micrograms/ml.

FIG. 46 shows the in vitro activity of MoA V_{H}CDRH₃ against P. aeruginosa ATCC 25853 strain. Amount of MoA V_{H}CDR₃ is in micrograms/ml.

FIG. 47 shows TNF-α production by peritoneal murine macrophages (PM) stimulated with MoA CDRs.

FIG. 48 shows IL-6 production by PM stimulated with MoA CDRs.

FIG. 49 shows TNF-α production by peritoneal murine neutrophils (PMN) stimulated with MoA CDRs.

FIG. 50 shows IL-6 production by PMN stimulated with MoA CDRs.

FIG. 51 shows MoA V_{H}CDR₃ uptake by different cell populations, namely dendritic, PM, PMN, and T cells.

FIGS. 52A and B shows the kinetics of biotin-labeled MoA V_{H}CDR uptake by PM. In FIG. 52A data are reported as the mean fluorescence intensity (MFI) (upper panel) and percentage of positive cells (lower panel). *, P<0.05 (b-VHCDR3-treated versus untreated cells, n=7). In FIG. 52B shows the uptake of b-VHCDR3 by PM by fluorescent microscopy.

FIG. 53 shows Phospho-Akt activation in murine macrophages stimulated with MoA V_{H}CDR₃. After incubation of murine macrophages for 1 hr in the presence or absence (NS) of MoA V_{H}CDR₃, LPS, negative control (NC) or irrelevant peptide (SP) (all at 10 micrograms/ml), cell lysates were subjected to Western blotting. Membranes were incubated with Abs to pAkt and Akt; pAkt was normalized against Akt (A)*, P<0.05 (treated vs untreated cells, n=5).

FIG. 54 shows the production of TNF-α in murine macrophages stimulated with MoA V_{H}CDR₃. After incubation of murine macrophages for 1 hr in the presence or absence (NS) of MoA V_{H}CDR₃, LPS, negative control (NC) or irrelevant peptide (SP) (all at 10 micrograms/ml), TNF-alpha level was evaluated in culture supernatants by specific ELISA assays; *, P<0.05 (treated versus untreated cells, n=5).

FIG. 55 shows phospho-IkBα activation in PM stimulated with MoA V_{H}CDR₃. Murine macrophages were incubated for 1 hr in the presence or absence (NS) of MoA V_{H}CDR₃, LPS, negative control (NC) or irrelevant peptide (SP) (all at 10 microgram/ml) with or without wortmannin. After incubation, cell lysates were subjected to Western blotting. Membranes were incubated with Abs to pIkBa and IkBα; pIkBα was normalized against IkBα. *, P<0.05 (treated versus untreated cells, n=5); +, P<0.05 (wortmannin-treated versus wortmannin-untreated cells, n=5).

FIG. 56 shows TNF-α gene expression in PM stimulated with MoA V_{H}CDR₃. Murine macrophages were cultured for 1, 6 and 18 hr as above described. After incubation, total RNA was isolated and analyzed for mRNA expression with RT-PCR. Transcript copy numbers were determined by qPCR. Copy numbers were normalized against the copy number of the GADPH gene (B). *, P<0.05 (treated versus untreated cells, n=5).

FIGS. 57A, B, and C show the expression of TLR-4 in PM stimulated with MoA V_{H}CDR₃. Specifically, FIG. 57A shows a graph depicting expression of TLR-4 in murine macrophages stimulated with MoA V_{H}CDR₃ for 1 and 6 hr in the presence or absence (NS) of MoA V_{H}CDR₃, LPS or negative control peptide (NC) (all 10 micrograms/ml). After incubation of murine macrophages, total RNA was isolated and analyzed for mRNA expression with RT-PCR. Transcript copy numbers normalized against the copy number of the GADPH. *, P<0.05 (VHCDR3 treated versus untreated cells, n=5). FIG. 57B shows induction of TLR4 by MoA V_{H}CDR₃. After incubation of murine macrophages, cell lysates were subjected to Western blotting. Membranes were incubated with Abs to TLR-4 and actin. TLR-4 production was normalized against actin. *, P>0.05 (MoA V_{H}CDR₃-treated versus untreated cells, n=5). Error bars, s.e.m. FIG. 57C shown expression of TLR-4 in murine macrophages stimulated with MoA V_{H}CDR₃ by FACScan flow cytometry. After incubation, permeabilized cells were reacted with RPE-labelled mAb to TLR-4 and analyzed by FACScan flow cytometry. Values represent the percentage of positive cells.

FIGS. 58A, B, C and D show the TNF-α induced TLR-4 expression in PM stimulated with MoA V_{H}CDR₃. Specifically, FIG. 58A shows TNF-α induction in murine macrophages in the presence or absence (NS) of MoA V_{H}CDR₃, VHCDR3, LPS or NC (all 10 micrograms/ml) for 1 hr. TNF-α level was evaluated in culture supernatants by specific ELISA assay. *, P<0.05 (treated versus untreated cells, n=7). FIG. 58B shows TNF-α induced TLR-4 expression in PM stimulated with MoA V_{H}CDR₃ by FACScan flow cytometry. Murine macrophages were cultured as in FIG. 61A and were then permeabilized cells and reacted with RPE-labeled mAb to TLR-4 and analyzed by FACScan flow cytometry. P<0.05 (VHCDR3 plus mAb to TNF-α treated versus VHCDR3 treated cells, n=7). FIG. 58C shows TNF-α induced TLR-4 expression by western blot in PM stimulated with MoA V_{H}CDR₃ as in FIG. 58A. After incubation, cell lysates were subjected to Western blotting. Membranes were incubated with Abs to TLR-4 and actin. TLR-4 production was normalized against actin (C)*, P<0.05 (VHCDR3 plus mAb to TNF-α treated versus VHCDR3 treated cells, n=5). FIG. 58D shows the expression level of TLR-4 gene by RT-PCR in murine macrophages in PM stimulated with MoA V_{H}CDR₃ as in FIG. 58A. Copy numbers were normalized against the copy number of the GADPH gene (D). *, P<0.05 (VHCDR3 plus mAb to TNF-α treated versus VHCDR3 treated cells, n=5). Error bars, s.e.m.

FIGS. 59A and B are graphs showing the in vitro activity of MoA V_{H}CDR₃ against systemic candidiasis. In FIG. 59A, the survival rate of subject mice is depicted. In FIG. 59B, the yeast recovery rate per kidney is graphed.

FIG. 60 is a pictoral graph depicting the mechanism of TLR-4 upregulation induced in PM by MoA V_{H}CDR₃.

FIG. 61 is a graph showing activity results of in vitro immunomodulatory activity of N10K and N10K asd on human monocytes.

FIGS. 62-72 are graphs depicting in vitro activity of each of the T11F alanine substituted peptides (T1A, C2A, R3A, V4A, D5A, H6A, R7A, G8A, L9A, T10A, F11A), respectively, against C. albicans SC5314 strain. *P<0.005, peptide treated versus untreated cells, t test. **P<0.05, peptide treated versus untreated cells, t test. Peptides C2A and R3A are shown tested at 50 and 100 micrograms/ml while the rest are tested at 2, 3, and 5 micrograms/ml.

FIG. 73 shows a graph of MoA V_{H}CDR₃ alanine substitutes affect of the peptide's ability to stimulate TNF alpha production by mouse peritoneal macrophages. FIG. 73A discloses SEQ ID NOS 4-13, respectively, in order of appearance.

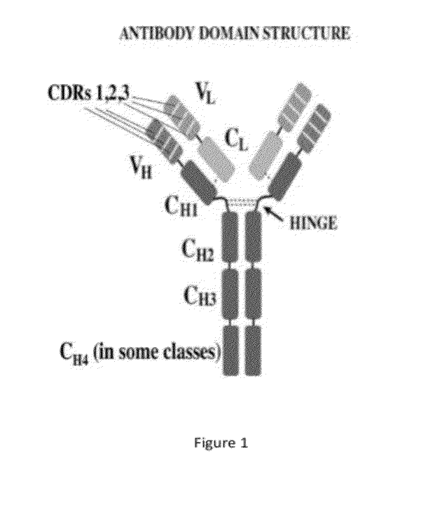

### DETAILED DESCRIPTION OF THE INVENTION

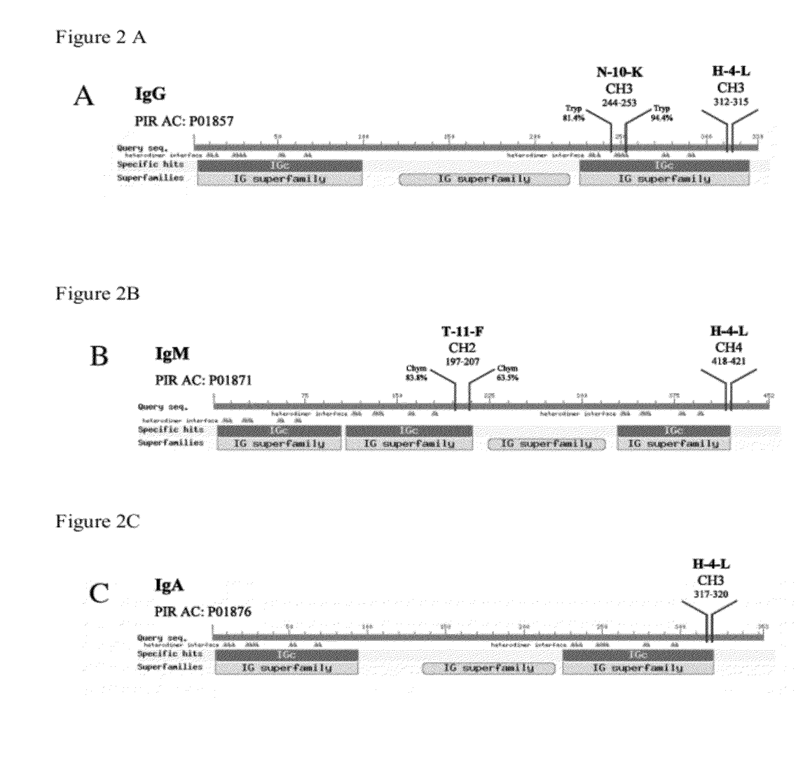

In a first embodiment, polypeptides are disclosed that exhibit a variety of activities comprising 1) antimicrobial, which includes antibacterial (antibacterial includes anti-Gram positive and anti-Gram negative bacteria), antifungal (antifungal includes antimold and antiyeast); 2) antiviral; 3) anticancer; and 4) immunomodulatory activities. In a related and preferred embodiment, the peptides are derived from C regions of Ig molecules. Of particularly preferred embodiment, the subject peptides can be found and identified in each of at least three different classes of Igs, namely, IgG, IgM, and IgA as shown in Table I below. Specifically, as disclosed in Table I and in FIG. 2, peptide N10K is derived from a C region of the IgG class Ig, peptide T11F is derived from a C region of the IgM class Ig, peptide H4L is derived from a C region of each of the IgG, IgA, and IgM class Igs, and the MoA VHCDR3, is derived from a CDR region of a murine mAb directed to difucosyl human blood group A substance.

TABLE I

| SEQ ID | Peptide | Description |
|---|---|---|
| 1 | N10K | (fragment of the C region of human |
| | | IgG molecules) |
| 14-23 | Alanine-scanning | Alanine substitutions of AA |
| | variants of SEQ ID 1 | within N10K |
| 24, 25 | Serine Amino acid | Serine substitutions of AA within |
| | variants of SEQ ID | N10K and alanine substitute of AA5 |
| | 1 and SEQ ID 18 | within N10K |
| 2 | T11F | (fragment of the C region of human |
| | | IgM molecules) |
| 27-37 | Alanine-scanning | Alanine substitutions |
| | variants of SEQ ID 2 | of AA within T11F |
| 3 | H4L | (fragment of the C region of human |
| | | and mammalian IgA, |
| | | IgM, and IgG molecules) |
| 4 | MoA V_{H}CDR₃ | (CDR₃ fragment of the H chain |
| | | of a murine IgM mAb directed |
| | | to difucosyl human blood group A |
| | | substance) |
| 5-13 | Alanine-scanning | Alanine substitutions of AA within |
| | variants of SEQ ID 4 | MoA V_{H}CDR₃ |

In a further preferred embodiment, the disclosed polypeptide sequences, possessing a broad spectrum of antimicrobial (including antibacterial which includes anti-Gram positive and anti-Gram negative bacteria, and antifungal, which includes antiyeast and antimold activities), antiviral, anticancer, and/or immunomodulatory activities, manifest their activities either by direct “cidal” action against the bacterium or fungus (Gram positive or Gram negative, mold or yeast) or through inhibition of virus replication or cancer cell growth or indirect action by immunomodulatory therapeutic action in the host mammal.

In a preferred embodiment, we disclose peptides derived from the C region of three Ig families, specifically, IgM, IgA, and IgG, each with at least one of antimicrobial, antiviral, anticancer and/or immunomodulatory activities, and a new class of peptides from a CDR region manifesting immunomodulatory activity.

In a further embodiment, the selection of peptides of the C region has been made according to Databank PIR “Protein Information Resource”: http://pir.georgetown.edu/. For the research of the sequence the following analyses has been performed by using the relative software: BLAST (blast.ncbi.nlm.nih.gov/Blast.cgi); multiple alignment with ClustalW (www.ebi.ac.uk/Tools/clustalw/); peptide cutter (expasy.org/tools/peptidecutter/) with cathepsins, trypsins and/or chymotrypsin-high specificity; calculation of Isoelectric Point (pI) with The Sequence Manipulation Suite 2 (www.bioinformatics.org/sms2/index.html). Based on the previous experience acquired in the study of biologically active CDR-related peptides, the definition of peptides of interest in the C region within each Ig class (FIG. 2) has been made according to: peptide length, proteolytic enzymes involved, percentage of cut, conserved amino acids (human Ig/different organisms), pI, and alternation of hydrophobic/hydrophilic residues in the sequence. The sequences, same or similar to those fragments which would be obtained upon digestion, might support the hypothesis that, beyond the half life of a typical Ig, fragments of the whole molecule may effectively influence the antiinfective and anticancer cellular immune response in a way reminiscent of regulatory peptides of innate immunity.

In a preferred embodiment, the CDR selections were made from the V region of a mouse mAb (IgM) specific for the difucosyl human blood group A substance (MoA) on the basis of the previously described sequences of VH and VL chain (Nickerson, 1995). Additionally, peptides of the invention include alanine-scanning variants peptides of the parent mouse MoA V_{H}CDR₃ peptide.

Table II shows the sequences of the peptides of the present invention.

TABLE II

| | Peptide amino acid | SEQ ID. |
|---|---|---|
| Peptide designation | sequence | No. |
| N10K | N Q V S L T C L V K | 1 |
| N10K alanine-scanning variant N1A | A Q V S L T C L V K | 14 |
| N10K alanine-scanning variant Q2A | N A V S L T C L V K | 15 |
| N10K alanine-scanning variant V3A | N Q A S L T C L V K | 16 |
| N10K alanine-scanning variant S4A | N Q V A L T C L V K | 17 |
| N10K alanine-scanning variant L5A | N Q V S A T C L V K | 18 |
| N10K alanine-scanning variant T6A | N Q V S L A C L V K | 19 |
| N10K alanine-scanning variant C7A | N Q V S L T A L V K | 20 |
| N10K alanine-scanning variant L8A | N Q V S L T C A V K | 21 |
| N10K alanine-scanning variant V9A | N Q V S L T C L A K | 22 |
| N10K alanine-scanning variant K10A | N Q V S L T C L V A | 23 |
| Consensus 4 motif Z-B-Hyd-Hyd, N10K | T C L V | 40 |
| Consensus 5 motif Hyd-X-Z-B-Hyd-Hyd, | V S L T C L | 43 |
| N10K | | |
| N10K serine variant L8S | N Q V S L T C S V K | 24 |
| N10K alanine/serine variant L5AL8S | N Q V S A T C S V K | 25 |
| T11F | T C R V D H R G L T F | 2 |
| T11F alanine-scanning variant T1A | A C R V D H R G L T F | 27 |
| T11F alanine-scanning variant C2A | T A R V D H R G L T F | 28 |
| T11F alanine-scanning variant R3A | T C A V D H R G L T F | 29 |
| T11F alanine-scanning variant V4A | T C R A D H R G L T F | 30 |
| T11F alanine-scanning variant D5A | T C R V A H R G L T F | 31 |
| T11F alanine-scanning variant H6A | T C R V D A R G L T F | 32 |
| T11F alanine-scanning variant R7A | T C R V D H A G L T F | 33 |
| T11F alanine-scanning variant G8A | T C R V D H R A L T F | 34 |
| T11F alanine-scanning variant L9A | T C R V D H R G A T F | 35 |
| T11F alanine-scanning variant T10A | T C R V D H R G L A F | 36 |
| T11F alanine-scanning variant F11A | T C R V D H R G L T A | 37 |
| Consensus 5 motif Hyd-X-Z-B-Hyd-Hyd, | V D H R G L | 42 |
| T11F | | |
| Consensus 4 motif Z-B-Hyd-Hyd, T11F | C R V D | 38 |
| Consensus 4 motif Z-B-Hyd-Hyd, T11F | H R G L | 39 |
| H4L | H E A L | 3 |
| MoA V_{H}CDR₃ | G Q Y G N L W F A Y | 4 |
| mAb MoA V_{H}CDR₃ alanine-scanning variant | A Q Y G N L W F A Y | 5 |
| 1 | | |
| mAb MoA V_{H}CDR₃ alanine-scanning variant | G A Y G N L W F A Y | 6 |
| 2 | | |
| mAb MoA V_{H}CDR₃ alanine-scanning variant | G Q A G N L W F A Y | 7 |
| 3 | | |
| mAb MoA V_{H}CDR₃ alanine-scanning variant | G Q Y A N L W F A Y | 8 |
| 4 | | |
| mAb MoA V_{H}CDR₃ alanine-scanning variant | G Q Y G A L W F A Y | 9 |
| 5 | | |
| mAb MoA V_{H}CDR₃ alanine-scanning variant | G Q Y G N A W F A Y | 10 |
| 6 | | |
| mAb MoA V_{H}CDR₃ alanine-scanning variant | G Q Y G N L A F A Y | 11 |
| 7 | | |
| mAb MoA V_{H}CDR₃ alanine-scanning variant | G Q Y G N L W A A Y | 12 |
| 8 | | |
| mAb MoA V_{H}CDR₃ alanine-scanning variant | G Q Y G N L W F A A | 13 |
| 9 | | |
| Consensus 4 motif Z-B-Hyd-Hyd, mAb | Q YG N | 41 |
| MoAV_{H}CDR₃ | | |
| Consensus 5 motif Hyd-X-Z-B-Hyd-Hyd, mAb | G N L W F A | 44 |
| MoAV_{H}CDR₃ | | |

In still a further embodiment, selected peptides of the C and V regions have been chemically synthesized and verified to possess antimicrobial, antiviral, anticancer and/or immunomodulatory activity. Moreover, recent data from CDR derived peptides, not here presented, implies a beta (β) sheet secondary structure for at least some of the C region peptides of interest. Such a structure may be common to immune competent C domain derived peptides. Moreover, we find that as exampled by the alanine substituted peptides Seq. Id. Nos. 5-23, and Seq. Id. Nos. 27-37, these peptides can manifest activity with at least a 10% change in their respective amino acid sequences from the naturally occurring peptide at almost any position. Further, as exampled by Seq. Id. No. 25, peptides can manifest activity with at least 20% change in their amino acid sequences from the naturally occurring peptide. Thus, we conceive and comprehend the claimed invention to include amino acid sequences possessing at least between 80% and 90% sequence identity with polypeptides derived from C and CDR regions of Igs possessing any of antibacterial, antifungal (antiyeast and antimold), anticancer, antiviral, and immunomodulatory activities. As shown in Table III below, the broad spectrum of activity is disclosed by the variety of pathogenic bacteria, fungi, virus, and cancer cells.

TABLE III

| Invention | Assay | | | Reference | |
|---|---|---|---|---|---|
| Peptide | type | Target | Genus and species | strain | Activity |
| N10K | In vitro | Fungus | Candida albicans | SC5314 | cidal |
| | In vivo | Fungus | C. albicans | CA-6 (virulent) | cidal |
| | In vitro | Fungus | C. albicans | UM4 | cidal |
| | | | | (caspofungin | |
| | | | | resistant) | |
| | In vitro | Fungus | Saccharomyces | YGR032W | cidal |
| | | | cerevisiae | (caspofungin | |
| | | | | resistant) | |
| | In vitro | Fungus | Cryptococcus | 6995 | cidal |
| | | | neoformans | | |
| | In vitro | Fungus | Malassezia furfur | 101 | cidal |
| | In vitro | Fungus | Aspergillus | 1163 | cidal |
| | | | fumigatus | | |
| | In vitro | Bacterium | Staphylococcus | 29213 | cidal |
| | | | aureus | | |
| | In vitro | Bacterium | Escherichia coli | ATCC25922 | cidal |
| | In vitro | Bacterium | Klebsiella | ATCC700603 | cidal |
| | | | pneumoniae | | |
| | In vitro | Bacterium | Pseudomonas | ATCC25853 | cidal |
| | | | aeruginosa | | |
| | In vitro | Virus | HIV-1 | | inhibitory |
| | In vitro | Cancer | B16F10-Nex2 | | inhibitory |
| | | cells | melanoma cell | | |
| | In vitro | Cancer | SKme128 melanoma | | inhibitory |
| | | cells | cell | | |
| | In vitro | Cancer | SKme125 melanoma | | inhibitory |
| | | cells | cell | | |
| | In vitro | Immune | Human monocytes | | Stimulation |
| | | cells | | | of IL- |
| | | | | | 6, Dectin- |
| | | | | | 1, IL- |
| | | | | | 12p40 |
| N10K | In vitro | Fungus | C. albicans | SC5314 | cidal |
| alanine | | | | | |
| substituted | | | | | |
| N10K | In vitro | Immune | Human monocytes | | Stimulation |
| alanine | | cells | | | of IL- |
| substituted | | | | | 6, Dectin- |
| | | | | | 1, IL- |
| | | | | | 12p40 |
| N10K | In vitro | Fungus | C. albicans | SC5314 | cidal |
| alanine/ | | | | | |
| serine | | | | | |
| double | | | | | |
| substituted | | | | | |
| T11F | In vitro | Fungus | C. albicans | SC5314 | cidal |
| | In vitro | Fungus | C. albicans | UM4 | cidal |
| | | | | (caspofungin | |
| | | | | resistant) | |
| | In vitro | Fungus | S. cerevisiae | YGR032W | cidal |
| | | | | (caspofungin | |
| | | | | resistant) | |
| | In vitro | Fungus | C. neoformans | 6995 | cidal |
| | In vitro | Fungus | M. furfur | 101 | cidal |
| | In vitro | Fungus | A. fumigatus | 1163 | cidal |
| | In vitro | Bacterium | S. aureus | 29213 | cidal |
| | In vitro | Bacterium | E. coli | ATCC25922 | cidal |
| | In vitro | Bacterium | K. pneumoniae | ATCC700603 | cidal |
| | In vitro | Bacterium | P. aeruginosa | ATCC25853 | cidal |
| | In vitro | Virus | HIV-1 | | inhibitory |
| | In vitro | Cancer | B16F10-Nex2 | | inhibitory |
| | | cells | melanoma cell | | |
| | | Cancer | SKme128 | | inhibitory |
| | | cells | melanoma cell | | |
| | | Cancer | SKme125 | | inhibitory |
| | | cells | melanoma cell | | |
| T11F | In vitro | Fungus | C. albicans | SC5314 | cidal |
| alanine | | | | | |
| derivatives | | | | | |
| H4L | In vitro | Fungus | C. albicans | SC5314 | cidal |
| | In vitro | Fungus | C. neoformans | 6995 | cidal |
| | In vitro | Fungus | M. furfur | 101 | cidal |
| | In vitro | Fungus | A. fumigatus | 1163 | cidal |
| | In vitro | Bacterium | S. aureus | 29213 | cidal |
| | In vitro | Bacterium | E. coli | ATCC25922 | cidal |
| | In vitro | Bacterium | K. pneumoniae | ATCC700603 | cidal |
| | In vitro | Bacterium | P. aeruginosa | ATCC25853 | cidal |
| | | Virus | HIV-1 | | inhibitory |
| MoAV_{H}CDR₃ | In vitro | Fungus | M. furfur | 101 | cidal |
| | In vitro | Fungus | A. fumigatus | 1163 | cidal |
| | In vitro | Bacterium | S. aureus | 29213 | cidal |
| | In vitro | Bacterium | E. coli | ATCC25922 | cidal |
| | In vitro | Bacterium | K. pneumoniae | ATCC700603 | cidal |
| | In vitro | Bacterium | P. aeruginosa | ATCC25853 | cidal |
| | In vitro | Immune | Murine | | Stimulation |
| | | cells | macrophages | | of |
| | | | | | TNF-α, |
| | | | | | IL-6 |
| | In vivo | Fungus | C. albicans | CA-6 | Indirectly |
| | | | | | cidal up |
| | | | | | regulation |
| | | | | | of TNF-α, |
| | | | | | TLR-4 |

### Activity Data for Invention Peptides is Presented Below in the Following Experimental Sections

### I. Example 1

### Data for N10K Peptide

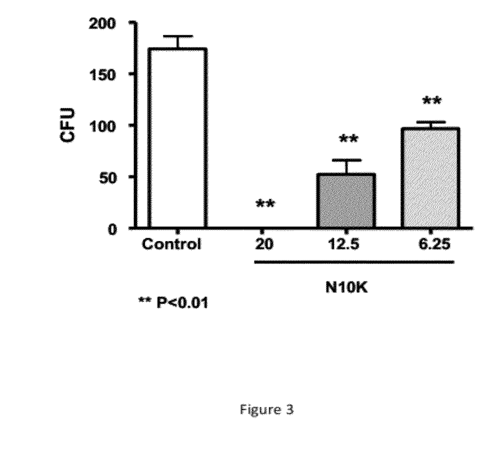

In vitro activity of N10K against C. albicans SC5314 strain. The candidacidal activity of N10K peptide against C. albicans was assessed by a conventional colony forming unit (CFU) assay as previously described (Polonelli et al., 2003; Manfredi et al., 2005). Briefly, cells of C. albicans SC5314 were incubated at 37° C. for 6 hours (hr) in the presence of N10K at the concentration indicated of 20, 12.5 or 6.25 microgram/ml, or in distilled water as control (C). After incubation, cell suspensions were plated on Sabouraud dextrose agar and incubated at 30° C. for 48 hours when CFU were counted (** P<0.01 for N10K treated versus untreated cells, by t test). As disclosed in FIG. 3, peptide N10K showed candidacidal activity in vitro against cells of C. albicans SC5314 strain. Based on several independent replications, an EC₅₀ of 10.04×10⁻⁶ mol/liter (95% confidence intervals 9.209-10.956×10⁻⁶) was determined.

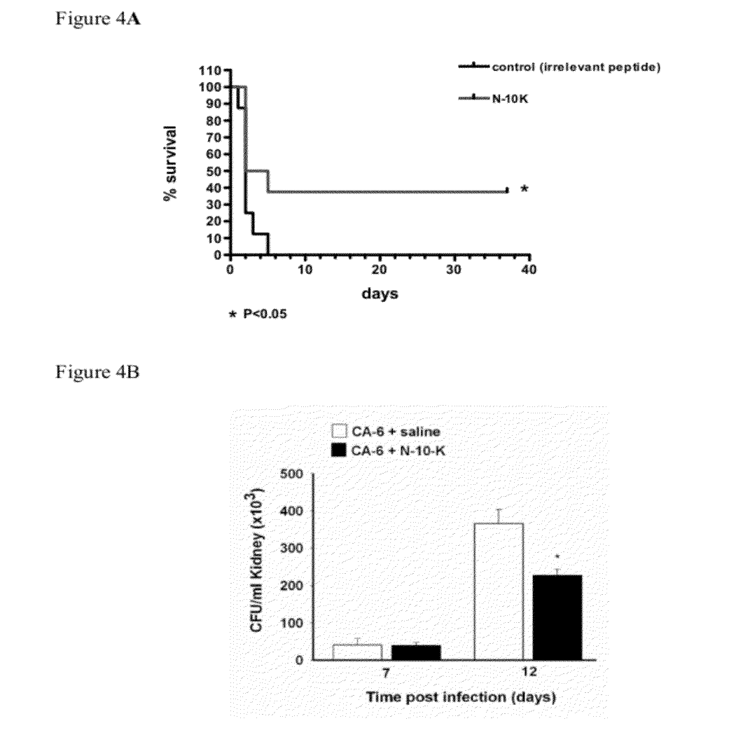

In vivo activity of N10K against systemic candidiasis caused in immunocompetent mice by the highly virulent C. albicans CA-6 strain. The anticandidal therapeutic activity of N10K was evaluated in a murine model of systemic candidiasis. Groups of 8 Balb/c mice were infected intravenously with 2×10⁶ cells of the highly virulent strain C. albicans CA-6 and given 50 micrograms of peptide N10K intraperitoneally 1, 24 and 48 hr after infection. Animals treated with an irrelevant peptide (SP) served as a negative control. Survival curves of infected mice were evaluated according to Mantel-Cox Logrank test and the difference between experimental and control groups resulted significant (* P<0.05 N10K versus SP treated mice) (FIG. 4A). CFU recovery from the kidneys of mice was determined 7 and 12 days after fungal infection. (* P<0.05 (N10K treated vs untreated mice) (FIG. 4B).

In vivo activity of N10K against vaginal candidiasis caused in mice by the fluconazole-susceptible C. albicans SA40 strain. The anticandidal therapeutic activity of N10K was evaluated in a murine model of vaginal candidiasis. Groups of 5 mice were injected subcutaneously with 0.02 mg of estradiol benzoate in 100 μl of saline solution, 48 hr yeast challenge and weekly thereafter. Then, mice given intravaginally 10⁶ cells of the fluconazole-susceptible C. albicans strain SA40 in 20 μl of saline solution on day 0 and were sampled for intravaginal CFU. N10K, 25 micrograms, was intravaginally administered in comparison to an irrelevant peptide (SP) as control, 1, 24 and 48 hours after the infectious challenge and sampled for intravaginal CFU at days 1, 2, 5, 7, 14, 21, and 28. The statistical significance was assessed by two-tailed Student's t test. On days 1, 2, 5, 7, 14, and 21, the differences in the CFU vaginal counts between N10K treated, untreated and SP treated animals were statistically significant (P<0.05) (Table V).

TABLE V

| Day | SA40 + N10K | SA40 + SP | SA40 |
|---|---|---|---|
| 0 | >100 | >100 | >100 |
| 1 | 72.6 ± 2.2* | >100 | >100 |
| 2 | 60 ± 1.9* | 96 ± 1.9 | 97 ± 1.3 |
| 5 | 50.4 ± 3.1* | 75.2 ± 2.1 | 74.4 ± 1.8 |
| 7 | 29.2 ± 0.8* | 40.2 ± 0.8 | 44.8 ± 1.9 |
| 14 | 12 ± 2.6* | 19.2 ± 1.2 | 22.2 ± 1.2 |
| 21 | 2.4 ± 1.3* | 9.4 ± 0.9 | 9.6 ± 0.9 |
| 28 | 0 | 0.8 ± 0.6 | 0.6 ± 0.4 |

*P < 0.05, N10K treated versus untreated and SP treated

In vivo activity of N10K against vaginal candidiasis caused in mice by the fluconazole-resistant C. albicans AIDS68 strain. The anticandidal therapeutic activity of N10K was evaluated in a murine model of vaginal candidiasis. Groups of 5 mice were injected subcutaneously with 0.02 mg of estradiol benzoate in 100 μl of saline solution, 48 hr yeast challenge and weekly thereafter. Then, mice given intravaginally 10⁶ cells of the fluconazole-resistant C. albicans strain AIDS68 in 20 μl of saline solution on day 0 and were sampled for intravaginal CFU. N10K, 25 micrograms, was intravaginally administered in comparison to an irrelevant peptide (SP) as control, 1, 24 and 48 hours after the infectious challenge and sampled for intravaginal CFU at days 1, 2, 5, 7, 14, 21, and 28. The statistical significance was assessed by two-tailed Student's t test. On days 1, 2, 5, 7, 14, and 21, the differences in the CFU vaginal counts between N10K treated, untreated, SP treated and fluconazole treated animals were statistically significant (P<0.05) (Table VI).

TABLE VI

| | | | AIDS68 + | |
|---|---|---|---|---|
| Day | AIDS68 + N10K | AIDS68 + SP | fluconazole | AIDS68 |
| 0 | >100 | >100 | >100 | >100 |
| 1 | 70.6 ± 1.7* | >100 | >100 | >100 |
| 2 | 55.4 ± 1.9* | 97 ± 1.9 | 93 ± 4.3 | >100 |
| 5 | 51.2 ± 2.1* | 65.6 ± 1.8 | 61 ± 2.5 | 66.8 ± 2.3 |
| 7 | 26.6 ± 1.7* | 45.2 ± 2.2 | 44 ± 2.9 | 47.2 ± 1.2 |
| 14 | 13 ± 1.3* | 25 ± 1.4 | 18.7 ± 3.8 | 25.8 ± 1.5 |
| 21 | 2.2 ± 1.1* | 7.8 ± 1.1 | 11.7 ± 0.7 | 12.6 ± 1.5 |
| 28 | 0 | 0.8 ± 0.3 | 0 | 0.8 ± 0.5 |

*P < 0.05 N10K treated versus untreated, SP and fluconazole treated

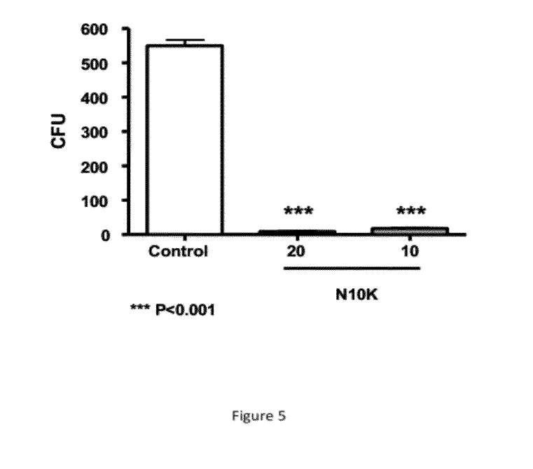

In vitro activity of N10K against caspofungin resistant C. albicans strain UM4. The candidacidal activity of N10K peptide against caspofungin resistant C. albicans strain has been evaluated by a conventional CFU assay (Polonelli et al., 2003). Briefly, Cells of C. albicans UM4, a clinical isolate from University of Milan, have been incubated at 37° C. for 6 hours in the presence of N-10-K at the concentration of 20 or 10 micrograms/ml, or in distilled water as control growth. After the incubation period, the cell suspensions have been plated on Sabouraud dextrose agar, then incubated at 30° C. and observed for CFU enumeration after 48 hours. As disclosed in FIG. 5, N10K showed fungicidal activity against a caspofungin resistant C. albicans strain (*** P<0.001, N-10-K treated versus untreated cells, t test).

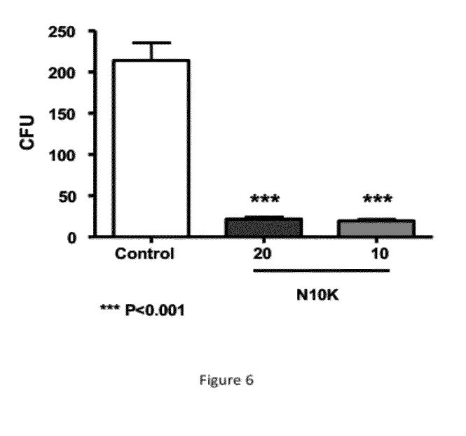

In vitro activity of N10K against a caspofungin-resistant S. cerevisiae strain YGR032W. The fungicidal activity of N10K peptide against a caspofungin-resistant S. cerevisiae strain has been evaluated by a conventional CFU assay (Conti et al., 2008). Briefly, Cells of S. cerevisiae YGR032W, a FSK2 deleted strain, have been incubated at 37° C. for 6 hours in the presence of N10K at the concentration of 20 or 10 micrograms/ml, or in distilled water as control growth. After the incubation period, the cell suspensions have been plated on Sabouraud dextrose agar, then incubated at 30° C. and observed for CFU enumeration after 48 hours. As disclosed in FIG. 6, N10K showed fungicidal activity against a caspofungin resistant strain of S. cerevisiae (*** P<0.001, N10K treated versus untreated cells, t test).

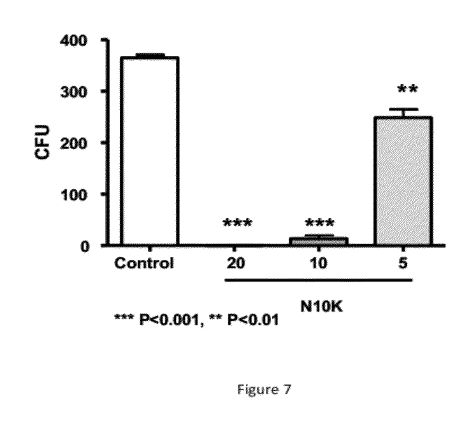

In vitro activity of N10K against C. neoformans 6995 strain. The fungicidal activity of N10K peptide against C. neoformans has been evaluated by a conventional CFU assay (Cenci et al., 2004). Cells of C. neoformans 6995 have been incubated at 37° C. for 6 hours in the presence of N10K at the concentration of 20, 10 or 5 micrograms/ml, or in distilled water as control growth. After the incubation period, the cell suspensions have been plated on Sabouraud dextrose agar, then incubated at 30° C. and observed for CFU enumeration after 48 hours. As disclosed in FIG. 7, N10K showed fungicidal activity against C. neoformans (*** P<0.001, ** P<0.01 N-10-K treated versus untreated cells, t test). The results of multiple experiments allowed the determination for the EC50 value as 5.155×10⁻⁶ mol/liter (95% confidence intervals 5.108-5.203×10⁻⁶).

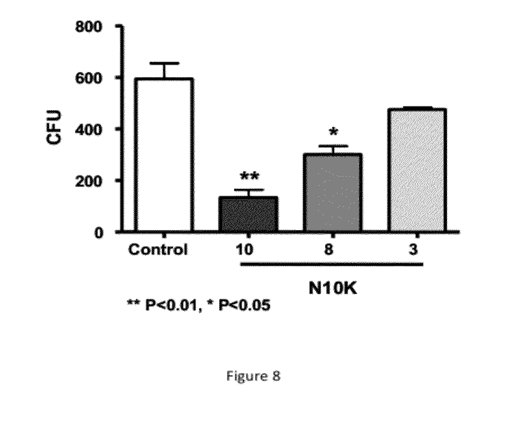

In vitro activity of N10K against M. furfur 101 strain. The microbicidal activity of N10K peptide against Malassezia furfur has been evaluated by a conventional CFU assay. Cells of M. furfur 101 have been incubated at 30° C. for 6 hours in the presence of N10K at the concentration of 10, 8 or 3 micrograms/ml, or in distilled water as control growth. After the incubation period, the cell suspensions were plated on Sabouraud dextrose agar added with Tween 20 (1%), then incubated at 30° C. and observed for CFU enumeration after 72 hours. As disclosed in FIG. 8, N10K showed fungicidal activity against M. furfur (** P<0.01, * P<0.05, N10K treated versus untreated cells, t test).

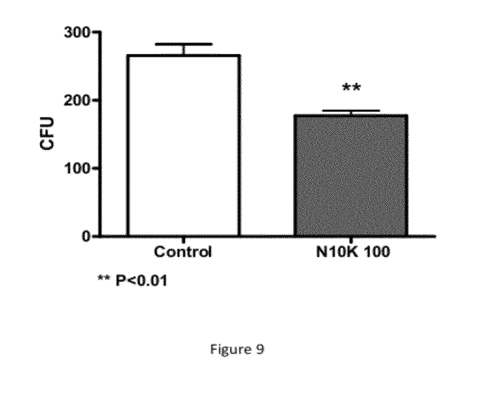

In vitro activity of N10K against A. fumigatus 1163 strain. The microbicidal activity of N10K peptide against A. fumigatus has been evaluated by a conventional CFU assay. Conidia of A. fumigatus 1163 have been incubated at 30° C. for 18 hours in the presence of N10K at the concentration of 100 micrograms/ml, or in distilled water as control growth. After the incubation period, the conidial suspensions have been plated on Sabouraud dextrose agar, then incubated at 30° C. and observed for CFU enumeration after 48 hours. As disclosed in FIG. 9, N10K showed fungicidal activity against A. fumigatus (** P<0.01, * P<0.05, treated versus untreated cells, t test).

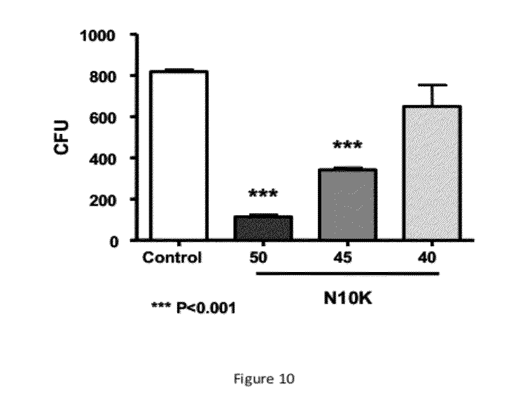

In vitro activity of N10K against S. aureus 29213 strain. The microbicidal activity of N10K peptide against S. aureus has been evaluated by a conventional CFU assay. Cells of S. aureus 29213 have been incubated at 37° C. for 5 hours in the presence of N10K at the concentration of 50, 45 or 40 micrograms/ml, or in distilled water as control growth. After the incubation period, the cell suspensions have been plated on Mueller Hinton agar, then incubated at 37° C. and observed for CFU enumeration after 24 hours. As disclosed in FIG. 10, N10 K showed fungicidal activity against S. aureus (*** P<0.001, N10K treated versus untreated cells, t test).

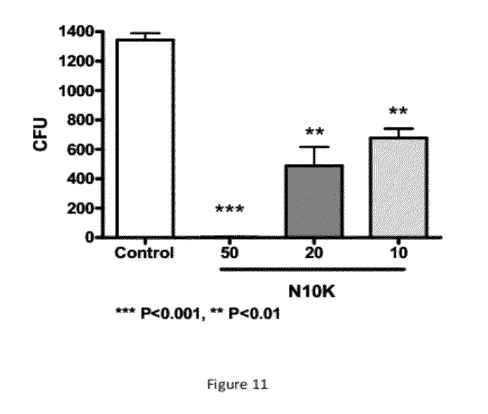

In vitro activity of N10K against E. coli ATCC 25922 strain. The bactericidal activity of N10K peptide against E. coli has been evaluated by a conventional CFU assay. Cells of E. coli ATCC 25922 have been incubated at 37° C. for 5 hours in the presence of N10K at the concentration of 50, 20 or 10 micrograms/ml, or in distilled water as control growth. After the incubation period, the cell suspensions have been plated on Mueller Hinton agar, then incubated at 37° C. and observed for CFU enumeration after 24 hours. As disclosed in FIG. 11, N10K showed bactericidal activity against E. coli (*** P<0.001, ** P<0.01, N10K treated versus untreated cells, t test).

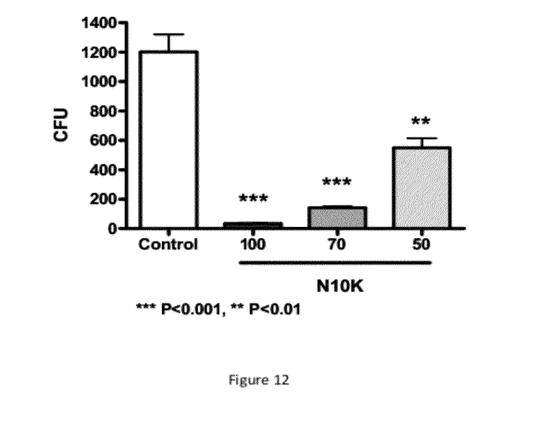

In vitro activity of N10K against K. pneumoniae ATCC 700603 strain. The bactericidal activity of N10K peptide against K. pneumoniae has been evaluated by a conventional CFU assay. Cells of K. pneumoniae ATCC 700603 have been incubated at 37° C. for 5 hours in the presence of N10K at the concentration of 100, 70 or 50 micrograms/ml, or in distilled water as control growth. After the incubation period, the cell suspensions have been plated on Mueller Hinton agar, then incubated at 37° C. and observed for CFU enumeration after 24 hours. As disclosed in FIG. 12, N10K showed bactericidal activity against K. pneumoniae (*** P<0.001, ** P<0.01, N10K treated versus untreated cells, t test).

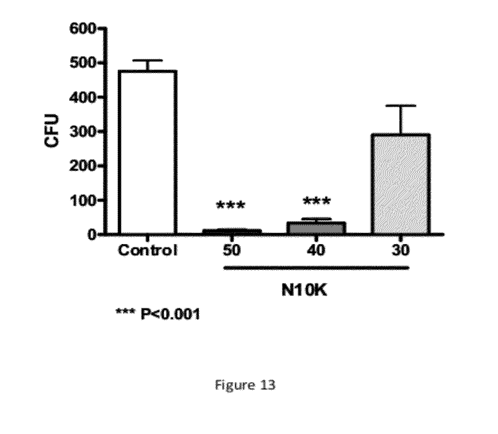

In vitro activity of N10K against P. aeruginosa ATCC 25853 strain. The bactericidal activity of N10K peptide against P. aeruginosa has been evaluated by a conventional CFU assay. Cells of P. aeruginosa ATCC 25853 have been incubated at 37° C. for 5 hours in the presence of N10K at the concentration of 50, 40 or 30 micrograms/ml, or in distilled water as control growth. After the incubation period, the cell suspensions have been plated on Mueller Hinton agar, then incubated at 37° C. and observed for CFU enumeration after 24 hours. As disclosed in FIG. 13, N10K showed bactericidal activity against P. aeruginosa (*** P<0.001, N10K treated versus untreated cells, t test).

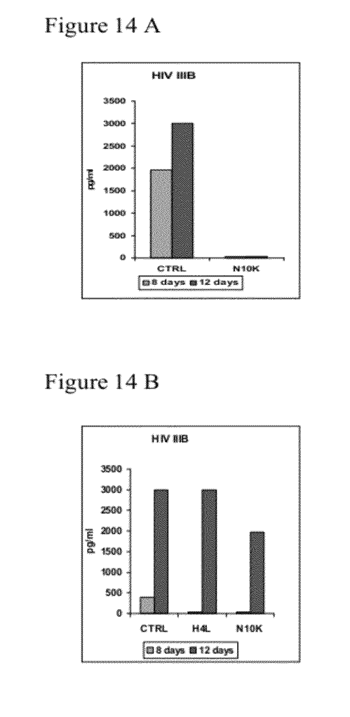

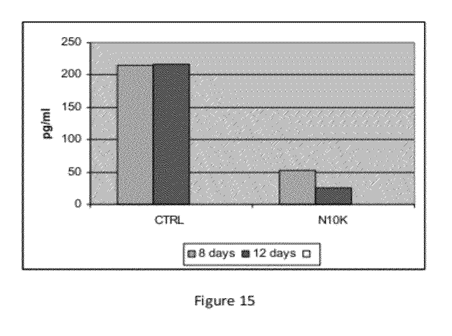

In vitro activity of N10K against HIV-1. PBMC from healthy donors were cultured at the concentration of 2×10⁶ cells/ml in 96 wells plates with RPMI 1640 supplemented with 10% FBS, 1% glutamine and 20 UI/ml rIL-2 for 24 hr before treatment with N10K, then the peptide was added at increasing concentrations (1, 10 and 20 micrograms/ml) for 24 hours. AlamarBlue was added (10% v/v) and incubated for 4 hours at 37° C. Cells viability was determined by the AlamarBlue Assay (Biosource International, Inc.). The absorbance was measured with an ELISA plate reader (Tecan Sunrise Absorbance Reader) at the double wavelength of 570/595 nm. AlamarBlue added to the complete RPMI1640 medium was used as blank. N10K demonstrated not to be cytotoxic for PBMC when it was employed in the 1-20 micrograms/ml concentration range, so the lowest and the intermediate doses of 1 and 10 micrograms/ml were elected for all the experiments. Moreover we could exclude the induction of apoptotic/necrotic effects on the U937 cell line performing the flow cytometry analysis after annexine-V/propidium iodide staining. To verify the anti-HIV-1 activity, an in vitro infection applying two different experimental approaches was performed. PBMC from 3 healthy donors were purified by Ficoll gradient centrifugation, mixed in pool and cultured in RPMI 1640 medium (10% FBS, 1% glutamine and 1% penicillin streptomycin). Before infection cells were stimulated for 24 hours with PHA (5 micrograms/ml) and after with rIL-2 (20 UI/ml). The first protocol consists in infecting PBMCs with HIV IIIB (X4) or BaL (R5) (0.5 m.o.i.) for two hours, washing twice and culturing in 96 wells plates for 12 days with 10 micrograms/ml of the compounds added together with rIL-2 every 4 days. The second protocol consists instead in pre-incubating PBMCs for two hours at 37° C. with the peptides (10 micrograms/ml), infecting with HIV IIIB or BaL (0.5 m.o.i.) for two hours, washing twice and culturing for 12 days. rIL-2 must be added to cells every 4 days (20 UI/ml). In both protocols PBMCs are collected at the days 8 and 12 of infection. Viral replication was evaluated measuring the concentration of the p24 antigen in the culture supernatants by the HIV p24 ELISA Ultrasensitive detection kit (PerkinElmer, Inc.). In the repeatedly performed in vitro infection assays the N10K peptide showed a significant antiviral activity against both the viral strains when employed in the concentration range of 1-10 micrograms/ml. No marked difference was noticed in the two concentrations induced effects. However, this viral inhibition was exclusively observed in PBMCs treated every 4 days after infection with the peptide (See FIGS. 14A and 15), whereas in the pre-incubation protocol the viral replication seem to be normal (R5 strain) or slightly, but not significantly, reduced (X4 strain) (See FIG. 14B). In comparison to the untreated controls, a relevant HIV inhibition was yet observed after 8 days of infection and this reduction continued to persist also at the 12^{th} day of culture. Of interest, when observed to the microscope, infected PBMCs appeared to cluster together after N10K treatment. To verify whether N10K induce a surface adhesion effect only in infected cells or also in the normal one, PBMC from tree healthy donors were purified by Ficoll gradient centrifugation, mixed in pool and cultured in RPMI 1640 medium (10% FBS, 1% glutamine and 1% penicillin streptomycin). Cells were seeded at the concentration of 500.000, 300.000 and 100.000 cells/well in a 96 wells plate for 7 days in presence of 20 UI/ml rIL-2 and 10 micrograms/ml peptides. The compounds and rIL-2 were added after 4 days of culture. At the 7th day of culture cells were observed at the microscope to analyze the aggregation effect. Interestingly, this attitude to aggregate in cluster wasn't parallelly observed in the infection controls and in the N10K treated uninfected PBMCs and it was exclusive for N10K but not for the other peptides. Concerning the possible mechanism of action by which the N10K peptide explains its antiviral activity, we firstly evaluated the syncytium formation attitude of N10K treated CD4+ cells in comparison to the untreated controls. CHO33T, HeLa ADA and HeLa LAI cell lines, which express constitutively the gp120 on their surface, were seeded at the concentration of 250.000 cells/well in 6 wells plate and cultured for 24 hr with D-MEM high glucose medium (10% FBS, 1% glutamine, 1% penicillin streptomycin and 1% Sodium Pyruvate; G418 was added to the CHO33T cells culture). The following day, CD4+T cells from 3 healthy donors were isolated by positive selection (Miltenyi Biotec Inc.), pooled together and co-cultured in complete RPMI1640 medium at the concentration of 1×10⁶ cells/well with the CHO33T and HeLa cell lines in presence of peptides (10 micrograms/ml). Untreated cells were used as controls. Syncytia formation was observed after 18 hours incubation at 37° C. The syncytium formation was evident both in controls and treated samples. This observation evidences that the N10K doesn't work on the interaction between the CD4 receptor and the viral gp120 (FIGS. 14 and 15).

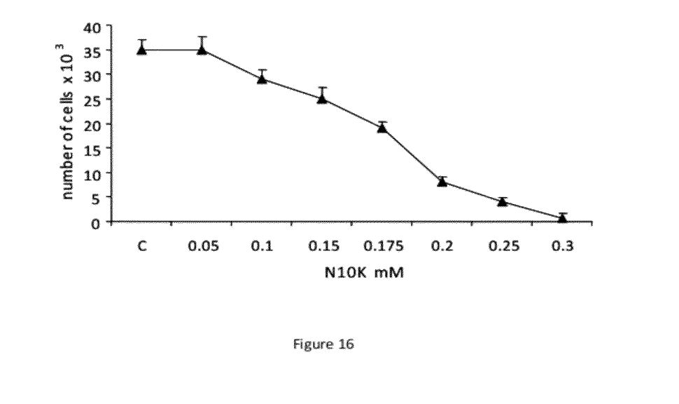

In vitro activity of N10K against B16F10-Nex2, SKme128 and SKme125 melanoma cells. Peptide N10K and the relative scramble peptide (SP) used as negative control were diluted from 1 mM to 0.05 mM in RPMI with 10% FCS and incubated with B16F10-Nex2, SKme128 and SKme125 cells (5×10³ cells/well) in 100 microliters per well for 12 hr at 37° C. Each peptide was tested in triplicate. After 12 hr, the cytotoxic activities of the peptides were determined by measuring cell viability by Trypan Blue exclusion. A 50% inhibition of cell growth was taken as a comparative index of cytotoxicity (EC₅₀). As disclosed in FIG. 16, N10K showed activity against B16F10-Nex2 melanoma cells. Similar results were obtained for SKme128 and SKme125 cells, data not here provided.

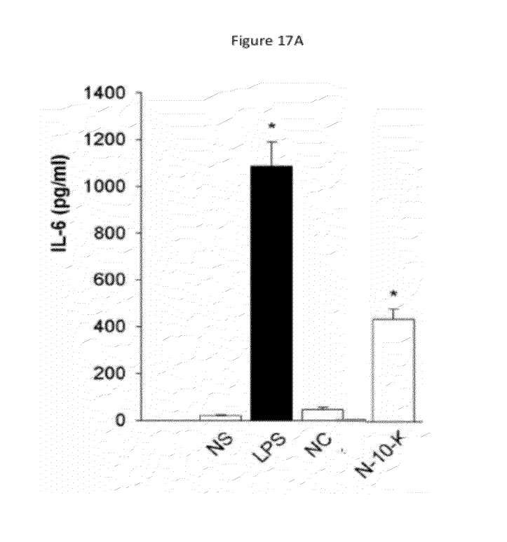

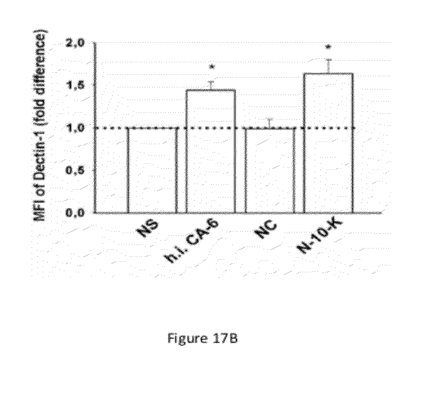

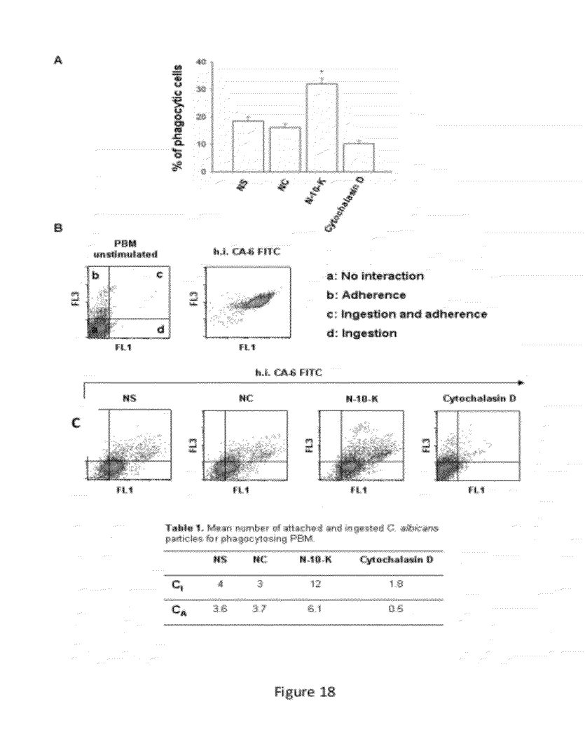

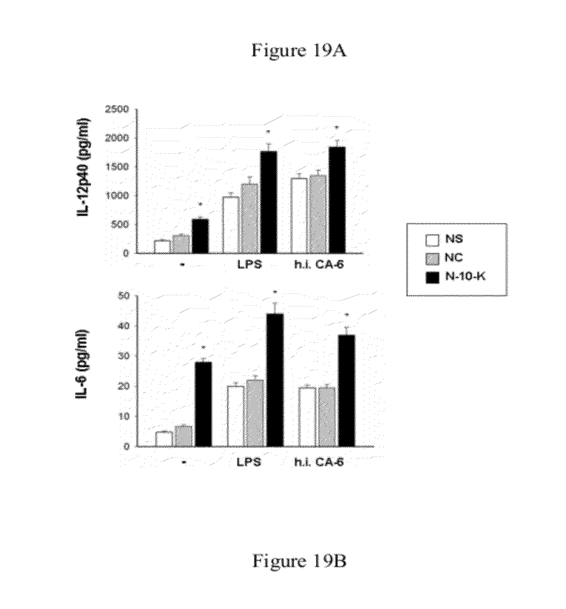

In vitro immunomodulatory activity of N10K on human immune cells. Human monocytes or PMN (both 10×10⁶/ml) were incubated in RPMI 1640 plus 10% FCS for 18 hr or 6 hr respectively in the presence or in the absence (NS) of LPS, negative control (NC) and peptides (all 10 micrograms/ml). After incubation culture supernatants were collected and tested for cytokines production by specific ELISA. FIG. 17A shows shows activity results of in vitro immunomodulatory activity of N10K on human monocytes IL-6 production. As disclosed in FIG. 17B, N10K stimulated the expression of Dectin-1 in human monocytes, incubated 30 minutes in presence or absence (NS) of heat inactivated C. albicans (h.i. CA-6) (E/T 1:10), NC or N10K (both 10 micrograms/ml). The phagocytosis of non opsonized h.i. CA-6 is shown in FIG. 18. Monocytes were incubated for 30 minutes in presence or absence (NS) of NC or N10K (both 10 micrograms/ml) or Cytochalasin D (30 μM). After incubation cells have been stimulated for 30 minutes with non opsonized h.i. CA-6 (E/T 1:10). The percentage of phagocytosis (FIG. 18A) and the medium number of yeast particles adhered or ingested by each monocyte (phagocytic index) (FIG. 18B) have been determined by cytofluorimetric analysis. In FIG. 18C is shown the mean number of attached and ingested C. albicans for phagocytosing PBM. Cytokines production induced by N10K is disclosed in FIG. 19. PBMC have been incubated 30 minutes in presence or absence (NS) of NC or N10K (both 10 micrograms/ml). After incubation PBMC have been stimulated for 18 h with LPS (10 micrograms/ml) or h.i. CA-6 (E/T 1:10). The levels of IL-12p40 and IL-6 have been evaluated in the supernatants by ELISA Kit.

### 2. Example II

### Data for T11F Peptide

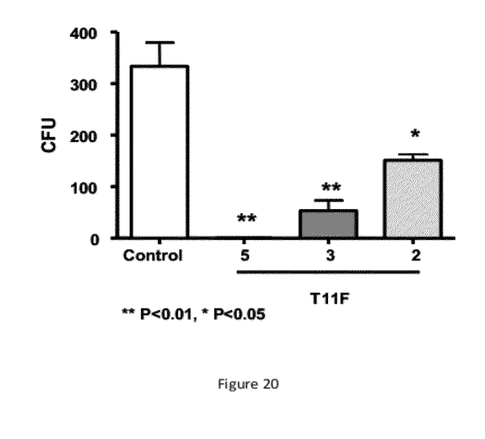

In vitro activity of T11F against C. albicans SC5314 strain. The candidacidal activity of T11F peptide against C. albicans was assessed by a conventional CFU assay. Cells of C. albicans SC5314 were incubated at 37° C. for 6 hours in the presence of T11F at the concentrations of 5, 3 or 2 micrograms/ml, or in distilled water as control (C). After incubation, cell suspensions were plated on Sabouraud dextrose agar and incubated at 30° C. for 48 hours when CFU were counted (** P<0.01, * P<0.05, T11F treated versus untreated cells, t test). As disclosed in FIG. 20, peptide T11F showed candidacidal activity in vitro against cells of C. albicans SC5314 strain. Based on several independent replications, an EC₅₀ of 1.599×10⁻⁶ mol/liter (95% confidence intervals 1.017-2.514×10⁻⁶) was determined.

In vivo activity of T11F against vaginal candidiasis caused in mice by the fluconazole-susceptible C. albicans SA40 strain. The anticandidal therapeutic activity of T11F was evaluated in a murine model of vaginal candidiasis. Groups of 5 mice were injected subcutaneously with 0.02 mg of estradiol benzoate in 100 μl of saline solution, 48 hr yeast challenge and weekly thereafter. Then, mice given intravaginally 10⁶ cells of the fluconazole-susceptible C. albicans strain SA40 in 20 μl of saline solution on day 0 and were sampled for intravaginal CFU. T11F, 25 micrograms, was intravaginally administered in comparison to an irrelevant peptide (SP) as control, 1, 24 and 48 hours after the infectious challenge and sampled for intravaginal CFU at days 1, 2, 5, 7, 14, 21, and 28. The statistical significance was assessed by two-tailed Student's t test. On days 1, 2, 5, 7, 14, and 21, the differences in the CFU vaginal counts between T11F treated, untreated and SP treated animals were statistically significant (P<0.05) (Table VII).

TABLE VII

| Day | SA40 + T11F | SA40 + SP | SA40 |
|---|---|---|---|
| 0 | >100 | >100 | >100 |
| 1 | 63.2 ± 2.1* | >100 | >100 |
| 2 | 54.8 ± 1.9* | 96 ± 1.9 | 97 ± 1.3 |
| 5 | 46.2 ± 1.9* | 75.2 ± 2.1 | 74.4 ± 1.8 |
| 7 | 24.8 ± 1.5* | 40.2 ± 0.8 | 44.8 ± 1.9 |
| 14 | 10.6 ± 1.2* | 19.2 ± 1.2 | 22.2 ± 1.2 |
| 21 | 2.2 ± 1* | 9.4 ± 0.9 | 9.6 ± 0.9 |
| 28 | 0 | 0.8 ± 0.6 | 0.6 ± 0.4 |

*P < 0.05, T11F treated versus untreated and SP treated

In vivo activity of T11F against vaginal candidiasis caused in mice by the fluconazole-resistant C. albicans AIDS68 strain. The anticandidal therapeutic activity of T11F was evaluated in a murine model of vaginal candidiasis. Groups of 5 mice were injected subcutaneously with 0.02 mg of estradiol benzoate in 100 μl of saline solution, 48 hr yeast challenge and weekly thereafter. Then, mice given intravaginally 10⁶ cells of the fluconazole-resistant C. albicans strain AIDS68 in 20 μl of saline solution on day 0 and were sampled for intravaginal CFU. T11F, 25 micrograms, was intravaginally administered in comparison to an irrelevant peptide (SP) as control, 1, 24 and 48 hours after the infectious challenge and sampled for intravaginal CFU at days 1, 2, 5, 7, 14, 21, and 28. The statistical significance was assessed by two-tailed Student's t test. On days 1, 2, 5, 7, 14, and 21, the differences in the CFU vaginal counts between T11F treated, untreated, SP treated and fluconazole treated animals were statistically significant (P<0.05) (Table VIII).

TABLE VIII

| | | | AIDS68 + | |
|---|---|---|---|---|
| Day | AIDS68 + T11F | AIDS68 + SP | fluconazole | AIDS68 |
| 0 | >100 | >100 | >100 | >100 |
| 1 | 62.2 ± 2* | >100 | >100 | >100 |
| 2 | 54.2 ± 2* | 97 ± 1.9 | 93 ± 4.3 | >100 |
| 5 | 53.2 ± 1.3* | 65.6 ± 1.8 | 61 ± 2.5 | 66.8 ± 2.3 |
| 7 | 21.2 ± 1* | 45.2 ± 2.2 | 44 ± 2.9 | 47.2 ± 1.2 |
| 14 | 10 ± 0.9* | 25 ± 1.4 | 18.7 ± 3.8 | 25.8 ± 1.5 |
| 21 | 1.8 ± 1.3* | 7.8 ± 1.1 | 11.7 ± 0.7 | 12.6 ± 1.5 |
| 28 | 0 | 0.8 ± 0.3 | 0 | 0.8 ± 0.5 |

*P < 0.05, T11F treated versus untreated, SP and fluconazole treated

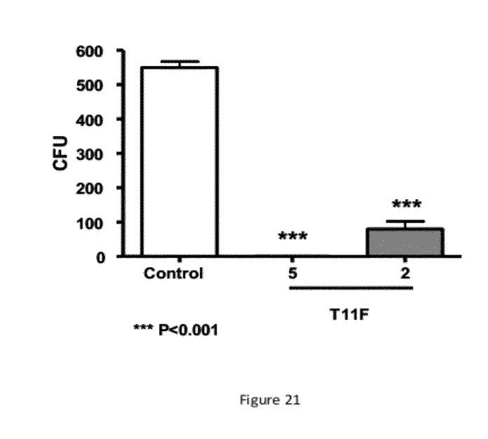

In vitro activity of T11F against caspofungin resistant C. albicans strain UM4. The candidacidal activity of T11F peptide against caspofungin resistant C. albicans strain has been evaluated by a conventional CFU assay. Cells of C. albicans UM4, a clinical isolate from University of Milan, have been incubated at 37° C. for 6 hours in the presence of T11F at the concentration of 5 or 2 micrograms/ml, or in distilled water as control growth. After the incubation period, the cell suspensions have been plated on Sabouraud dextrose agar, then incubated at 30° C. and observed for CFU enumeration after 48 hours. As disclosed in FIG. 21, T11F showed candidacidal activity against a caspofungin resistant C. albicans strain (*** P<0.001, T-11-F treated versus untreated cells, t test).

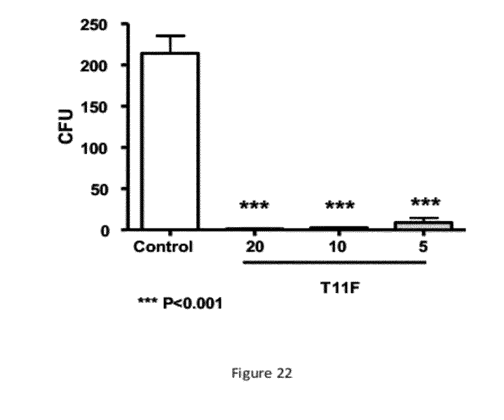

In vitro activity of T11F against a caspofungin-resistant S. cerevisiae strain YGR032W. The fungicidal activity of T11F peptide against a caspofungin-resistant S. cerevisiae strain has been evaluated by a conventional CFU assay. Cells of S. cerevisiae YGR032W, a FSK2 deleted strain, have been incubated at 37° C. for 6 hours in the presence of T11F at the concentration of 20, 10 or 5 micrograms/ml, or in distilled water as control growth. After the incubation period, the cell suspensions have been plated on Sabouraud dextrose agar, then incubated at 30° C. and observed for CFU enumeration after 48 hours. As disclosed in FIG. 22, T11F showed fungicidal activity against a caspofungin-resistant S. cerevisiae strain (*** P<0.001, T-11-F treated versus untreated cells, t test).

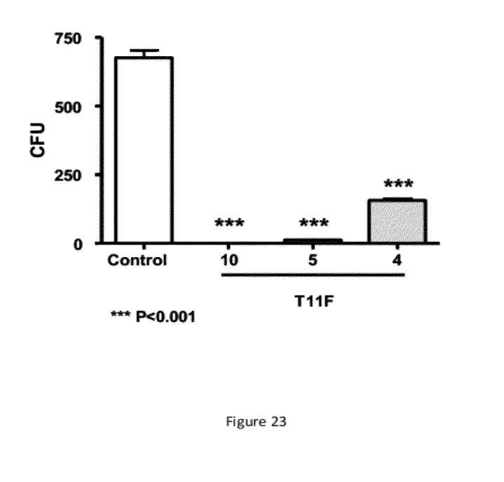

In vitro activity of T11F against C. neoformans 6995 strain. The fungicidal activity of T11F peptide against C. neoformans has been evaluated by a conventional CFU assay. Cells of C. neoformans 6995 have been incubated at 37° C. for 6 hours in the presence of T11F at the concentration of 10, 5 or 4 micrograms/ml, or in distilled water as control growth. After the incubation period, the cell suspensions have been plated on Sabouraud dextrose agar, then incubated at 30° C. and observed for CFU enumeration after 48 hours. As disclosed in FIG. 23, T11F showed fungicidal activity against C. neoformans (*** P<0.0001, T-11-F treated versus untreated cells, t test). The results of multiple experiments allowed the determination for the EC₅₀ value as 2.693×10⁻⁶ mol/liter (95% confidence intervals 2.692-2.694×10⁻⁶).

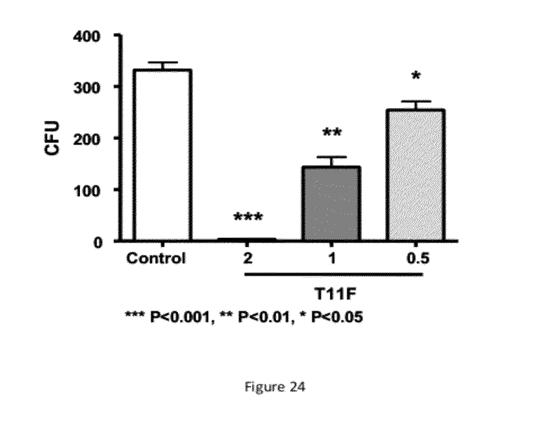

In vitro activity of T11F against M. furfur 101 strain. The fungicidal activity of T11F peptide against M. furfur has been evaluated by a CFU assay. Cells of M. furfur 101 have been incubated at 30° C. for 6 hours in the presence of T11F at the concentration of 2, 1 or 0.5 micrograms/ml, or in distilled water as control growth. After the incubation period, the cell suspensions have been plated on Sabouraud dextrose agar added with Tween 20 (1%), then incubated at 30° C. and observed for CFU enumeration after 72 hours. As disclosed in FIG. 24, T11F showed fungicidal activity against M. furfur (*** P<0.001, ** P<0.01, * P<0.05, T11F treated versus untreated cells, t test).

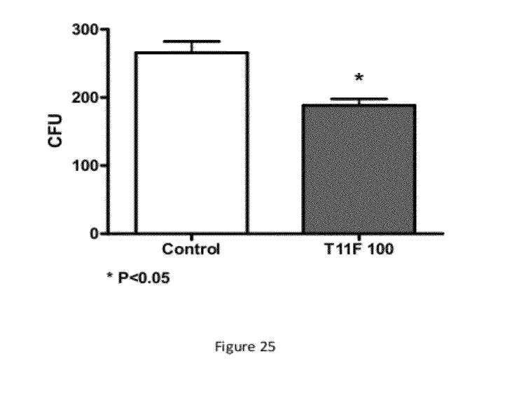

In vitro activity of T11F against A. fumigatus 1163 strain. The fungicidal activity of T11F peptide against A. fumigatus has been evaluated by a conventional CFU assay. Conidia of A. fumigatus 1163 have been incubated at 30° C. for 18 hours in the presence of T11F at the concentration of 100 micrograms/ml, or in distilled water as control growth. After the incubation period, the conidial suspensions have been plated on Sabouraud dextrose agar, then incubated at 30° C. and observed for CFU enumeration after 48 hours. As disclosed in FIG. 25, T11F showed fungicidal activity against A. fumigatus (* P<0.05, treated versus untreated cells, t test).

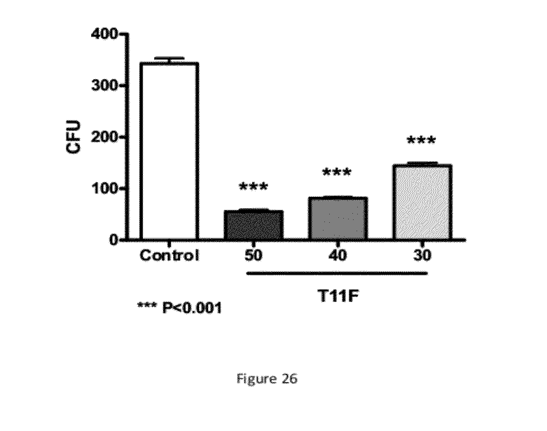

In vitro activity of T11F against S. aureus 29213 strain. The bactericidal activity of T11F peptide against S. aureus has been evaluated by a conventional CFU assay. Cells of S. aureus 29213 have been incubated at 37° C. for 5 hours in the presence of T11F at the concentration of 50, 40 or 30 micrograms/ml, or in distilled water as control growth. After the incubation period, the cell suspensions have been plated on Mueller Hinton agar, then incubated at 37° C. and observed for CFU enumeration after 24 hours. As disclosed in FIG. 26, T11F showed bactericidal activity against S. aureus (*** P<0.001, T11F treated versus untreated cells, t test).

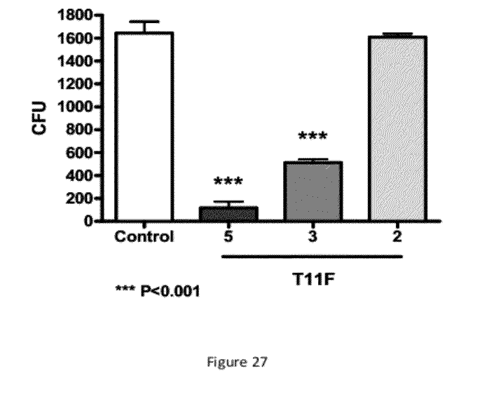

In vitro activity of T11F against E. coli ATCC 25922 strain. The bactericidal activity of T11F peptide against E. coli has been evaluated by a conventional CFU assay. Cells of E. coli ATCC 25922 have been incubated at 37° C. for 5 hours in the presence of T11F at the concentration of 5, 3 or 2 micrograms/ml, or in distilled water as control growth. After the incubation period, the cell suspensions have been plated on Mueller Hinton agar, then incubated at 37° C. and observed for CFU enumeration after 24 hours. As disclosed in FIG. 27, T11F showed bactericidal activity against E. coli (*** P<0.001, T11F treated versus untreated cells, t test).

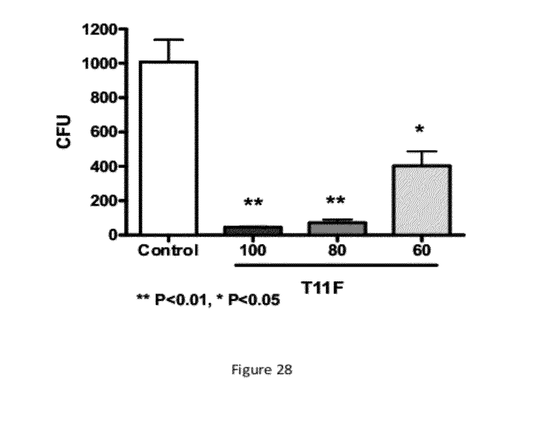

In vitro activity of T11F against K. pneumoniae ATCC 700603 strain. The bactericidal activity of T11F peptide against K. pneumoniae has been evaluated by a CFU assay. Cells of K. pneumoniae ATCC 700603 have been incubated at 37° C. for 5 hours in the presence of T11F at the concentration of 100, 80 or 60 micrograms/ml, or in distilled water as control growth. After the incubation period, the cell suspensions have been plated on Mueller Hinton agar, then incubated at 37° C. and observed for CFU enumeration after 24 hours. As disclosed in FIG. 28, T11F showed bactericidal activity against K. pneumoniae (** P<0.01, * P<0.05 T11F treated versus untreated cells, t test).

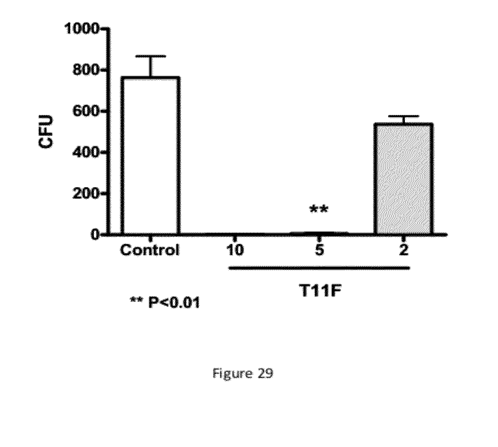

In vitro activity of T11F against P. aeruginosa ATCC 25853 strain. The bactericidal activity of T11F peptide against P. aeruginosa has been evaluated by a conventional CFU assay. Cells of P. aeruginosa ATCC 25853 have been incubated at 37° C. for 5 hours in the presence of T11F at the concentration of 10, 5 or 2 micrograms/ml, or in distilled water as control growth. After the incubation period, the cell suspensions have been plated on Mueller Hinton agar, then incubated at 37° C. and observed for CFU enumeration after 24 hours. As disclosed in FIG. 29, T11F showed bactericidal activity against P. aeruginosa (** P<0.01, T11F treated versus untreated cells, t test).

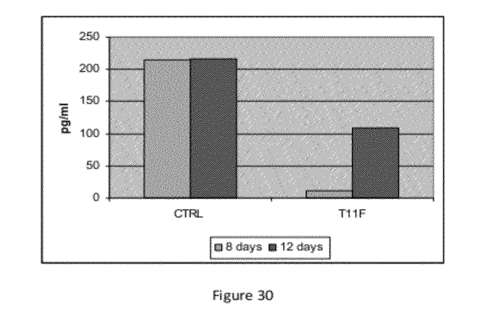

In vitro activity of T11F against HIV-1. PBMC from healthy donors were cultured at the concentration of 2×10⁶ cells/ml in 96 wells plates with RPMI 1640 supplemented with 10% FBS, 1% glutamine and 20 UI/ml rIL-2 for 24 hr before treatment with T11F, then the peptide was added at increasing concentrations (1, 10 and 20 micrograms/ml) for 24 hours. AlamarBlue was added (10% v/v) and incubated for 4 hours at 37° C. Cells viability was determined by the AlamarBlue Assay (Biosource International, Inc.). The absorbance was measured with an ELISA plate reader (Tecan Sunrise Absorbance Reader) at the double wavelength of 570/595 nm. AlamarBlue added to the complete RPMI1640 medium was used as blank. T11F demonstrated not to be cytotoxic for PBMC when it was employed in the 1-20 micrograms/ml concentration range, so the lowest and the intermediate doses of 1 and 10 micrograms/ml were elected for all the experiments. Moreover we could exclude the induction of apoptotic/necrotic effects on the U937 cell line performing the flow cytometry analysis after annexine-V/propidium iodide staining. To verify the anti-HIV-1 activity, an in vitro infection applying two different experimental approaches was performed. PBMC from 3 healthy donors were purified by Ficoll gradient centrifugation, mixed in pool and cultured in RPMI 1640 medium (10% FBS, 1% glutamine and 1% penicillin streptomycin). Before infection cells were stimulated for 24 hours with PHA (5 micrograms/ml) and after with rIL-2 (20 UI/ml). The first protocol consists in infecting PBMCs with HIV IIIB (X4) or BaL (R5) (0.5 m.o.i.) for two hours, washing twice and culturing in 96 wells plates for 12 days with 10 micrograms/ml of the compounds added together with rIL-2 every 4 days. The second protocol consists instead in pre-incubating PBMCs for two hours at 37° C. with the peptides (10 micrograms/ml), infecting with HIV IIIB or BaL (0.5 m.o.i.) for two hours, washing twice and culturing for 12 days. rIL-2 must be added to cells every 4 days (20 UI/ml). In both protocols PBMCs are collected at the days 8 and 12 of infection. Viral replication was evaluated measuring the concentration of the p24 antigen in the culture supernatants by the HIV p24 ELISA Ultrasensitive detection kit (PerkinElmer, Inc.). T11F keep low the BaL replication only if it was supplied every 4 days after infection (FIG. 30).

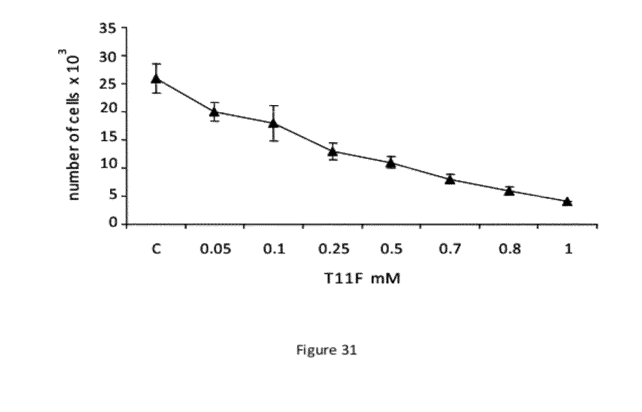

In vitro activity of T11F against B16F10-Nex2, SKme128 and SKme125 melanoma cells. Peptide T11F and the relative scramble peptide (SP) used as negative control were diluted from 1 mM to 0.05 mM in RPMI with 10% FCS and incubated with B16F10-Nex2, SKme128 and SKme125 cells (5×10³ cells/well) in 100 microL per well for 12 hr at 37 u° C. Each peptide was tested in triplicate. After 12 hr, the cytotoxic activities of the peptides were determined by measuring cell viability by Trypan Blue exclusion. A 50% inhibition of cell growth was taken as a comparative index of cytotoxicity (EC₅₀). As disclosed in FIG. 31, T11F showed activity against B16F10-Nex2 melanoma cells. Similar results, not here provided, were found with both SKme128 and SKme125 cells.

### 3. Experiment III

### Data for H4L Peptide

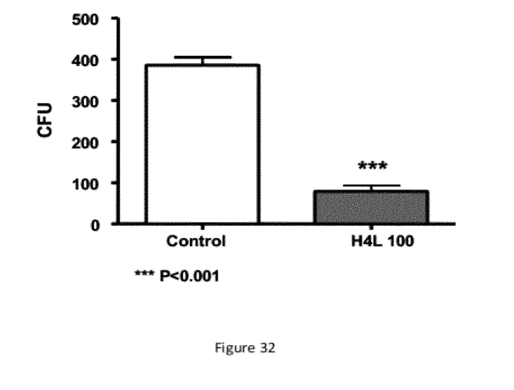

In vitro activity of H4L against C. albicans SC5314 strain. The candidacidal activity of H4L peptide against C. albicans has been evaluated by a conventional CFU assay. Cells of C. albicans SC5314 have been incubated at 37° C. for 6 hours in the presence of H4L at the concentration of 100 micrograms/ml, or in distilled water as control growth. After the incubation period, the cell suspensions have been plated on Sabouraud dextrose agar, then incubated at 30° C. and observed for CFU enumeration after 48 hours. As disclosed in FIG. 32, H4L showed candidacidal activity against C. albicans (*** P<0.001, treated versus untreated cells, t test).

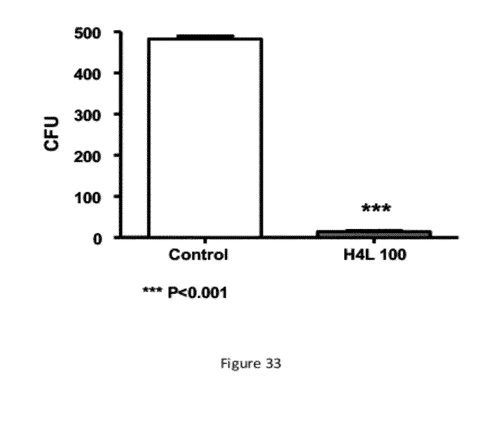

In vitro activity of H4L against C. neoformans 6995 strain. The fungicidal activity of H4L peptide against C. neoformans has been evaluated by a conventional CFU assay. Cells of C. neoformans 6995 have been incubated at 37° C. for 6 hours in the presence of H4L at the concentration of 100 micrograms/ml, or in distilled water as control growth. After the incubation period, the cell suspensions have been plated on Sabouraud dextrose agar, then incubated at 30° C. and observed for CFU enumeration after 48 hours. As disclosed in FIG. 33, H4L showed fungicidal activity against C. neoformans (*** P<0.001, treated versus untreated cells, t test).

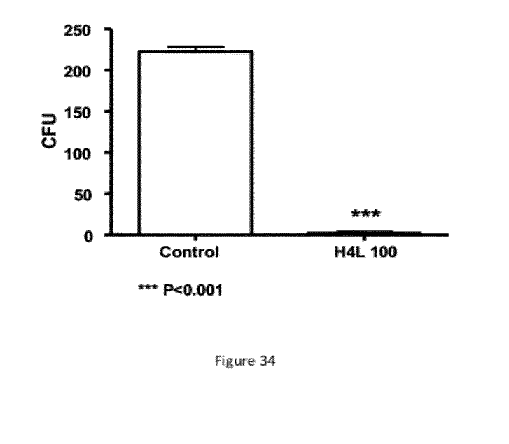

In vitro activity of H4L against M. furfur 101 strain. The fungicidal activity of H4L peptide against M. furfur has been evaluated by a conventional CFU assay. Cells of M. furfur 101 have been incubated at 30° C. for 6 hours in the presence of peptides at the concentration of 100 micrograms/ml, or in distilled water as control growth. After the incubation period, the cell suspensions have been plated on Sabouraud dextrose agar added with Tween 20 (1%), then incubated at 30° C. and observed for CFU enumeration after 72 hours. As disclosed in FIG. 34, H4L showed fungicidal activity against M. furfur (*** P<0.001, treated versus untreated cells, t test).

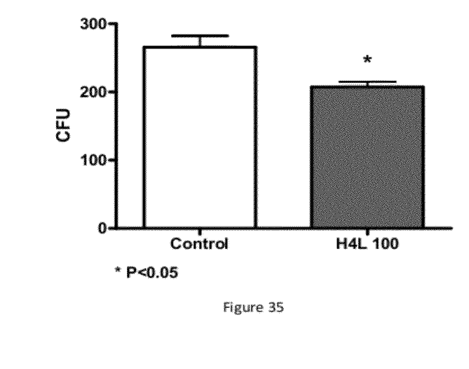

In vitro activity of H4L against A. fumigatus 1163 strain. The fungicidal activity of H4L peptide against A. fumigatus has been evaluated by a CFU assay. Conidia of A. fumigatus 1163 have been incubated at 30° C. for 18 hours in the presence of H4L at the concentration of 100 micrograms/ml, or in distilled water as control growth. After the incubation period, the cell suspensions have been plated on Sabouraud dextrose agar, then incubated at 30° C. and observed for CFU enumeration after 48 hours. As disclosed in FIG. 35, H4L showed fungicidal activity against A. fumigatus (* P<0.05, treated versus untreated cells, t test).

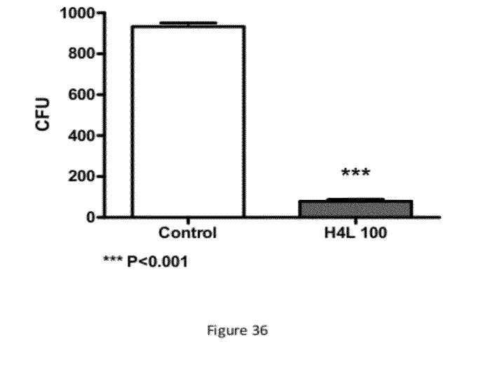

In vitro activity of H4L against S. aureus 29213 strain. The bactericidal activity of H4L peptide against S. aureus has been evaluated by a conventional CFU) assay. Cells of S. aureus 29213 have been incubated at 37° C. for 5 hours in the presence of peptides at the concentration of 100 micrograms/ml, or in distilled water as control growth. After the incubation period, the cell suspensions have been plated on Mueller Hinton agar, then incubated at 37° C. and observed for CFU enumeration after 24 hours. As disclosed in FIG. 36, H4L showed bactericidal activity against S. aureus (*** P<0.001, treated versus untreated cells, t test).

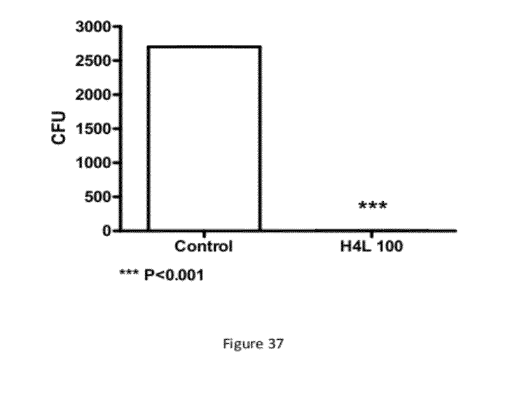

In vitro activity of H4L against E. coli ATCC 25922 strain. The bactericidal activity of H4L peptide against E. coli has been evaluated by a conventional CFU assay. Cells of E. coli ATCC 25922 have been incubated at 37° C. for 5 hours in the presence of H4L at the concentration of 100 micrograms/ml, or in distilled water as control growth. After the incubation period, the cell suspensions have been plated on Mueller Hinton agar, then incubated at 37° C. and observed for CFU enumeration after 24 hours. As disclosed in FIG. 37, H4L showed bactericidal activity against E. coli (*** P<0.001, treated versus untreated cells, t test).

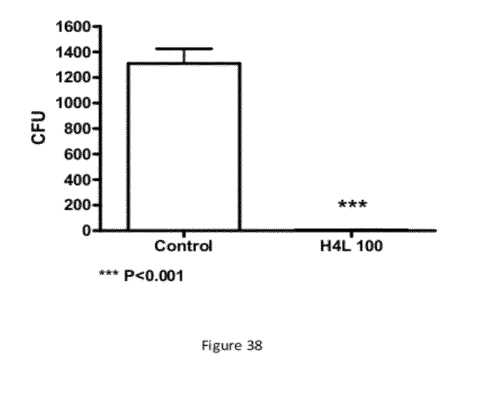

In vitro activity of H4L against K. pneumoniae ATCC 700603 strain. The bactericidal activity of H4L peptide against K. pneumoniae has been evaluated by a conventional CFU assay. Cells of K. pneumoniae ATCC 700603 have been incubated at 37° C. for 5 hours in the presence of H4L at the concentration of 100 micrograms/ml, or in distilled water as control growth. After the incubation period, the cell suspensions have been plated on Mueller Hinton agar, then incubated at 37° C. and observed for CFU enumeration after 24 hours. As disclosed in FIG. 38, H4L showed bactericidal activity against K. pneumoniae (*** P<0.001, treated versus untreated cells, t test).

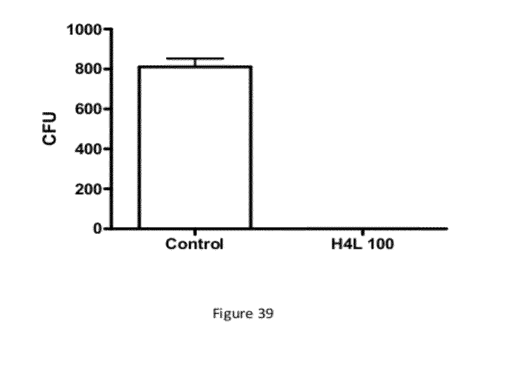

In vitro activity of H4L against P. aeruginosa ATCC 25853 strain. The microbicidal activity of H4L peptide against P. aeruginosa has been evaluated by a conventional CFU assay. Cells of P. aeruginosa ATCC 25853 have been incubated at 37° C. for 5 hours in the presence of H4L at the concentration of 100 micrograms/ml, or in distilled water as control growth. After the incubation period, the cell suspensions have been plated on Mueller Hinton agar, then incubated at 37° C. and observed for CFU enumeration after 24 hours. As disclosed in FIG. 39, H4L showed bactericidal activity against P. aeruginosa.

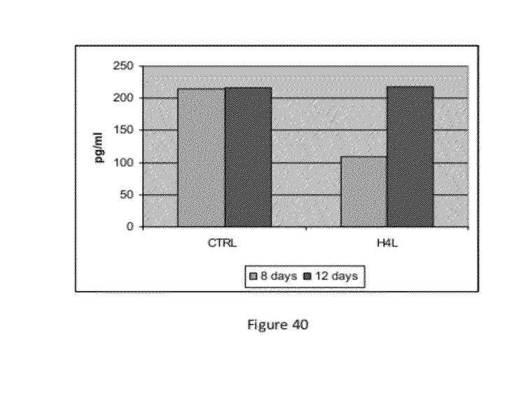

In vitro activity of H4L against HIV-1. PBMC from healthy donors were cultured at the concentration of 2×10⁶ cells/ml in 96 wells plates with RPMI 1640 supplemented with 10% FBS, 1% glutamine and 20 UI/ml rIL-2 for 24 hr before treatment with H41, then the peptide was added at increasing concentrations (1, 10 and 20 micrograms/ml) for 24 hours. AlamarBlue was added (10% v/v) and incubated for 4 hours at 37° C. Cells viability was determined by the AlamarBlue Assay (Biosource International, Inc.). The absorbance was measured with an ELISA plate reader (Tecan Sunrise Absorbance Reader) at the double wavelength of 570/595 nm. AlamarBlue added to the complete RPMI1640 medium was used as blank. H4L demonstrated not to be cytotoxic for PBMC when it was employed in the 1-20 micrograms/ml concentration range, so the lowest and the intermediate doses of 1 and 10 micrograms/ml were elected for all the experiments. Moreover we could exclude the induction of apoptotic/necrotic effects on the U937 cell line performing the flow cytometry analysis after annexine-V/propidium iodide staining. To verify the anti-HIV-1 activity, an in vitro infection applying two different experimental approaches was performed. PBMC from 3 healthy donors were purified by Ficoll gradient centrifugation, mixed in pool and cultured in RPMI 1640 medium (10% FBS, 1% glutamine and 1% penicillin streptomycin). Before infection cells were stimulated for 24 hours with PHA (5 micrograms/ml) and after with rIL-2 (20 UI/ml). The first protocol consists in infecting PBMCs with HIV IIIB (X4) or BaL (R5) (0.5 m.o.i.) for two hours, washing twice and culturing in 96 wells plates for 12 days with 10 micrograms/ml of the compounds added together with rIL-2 every 4 days. The second protocol consists instead in pre-incubating PBMCs for two hours at 37° C. with the peptide (10 micrograms/ml), infecting with HIV IIIB or BaL (0.5 m.o.i.) for two hours, washing twice and culturing for 12 days. rIL-2 must be added to cells every 4 days (20 UI/ml). In both protocols PBMCs are collected at the days 8 and 12 of infection. Viral replication was evaluated measuring the concentration of the p24 antigen in the culture supernatants by the HIV p24 ELISA Ultrasensitive detection kit (PerkinElmer, Inc.). In fact, the H4L peptide administered to cell prior to the viral infection seemed to control HIV IIIB replication only during the first 8 days of culture, and kept low the BaL replication till the day 8 of infection only if it was supplied every 4 days after infection (FIG. 40).

### 4. Experiment IV

### Data for MoA VhCDR₃ Peptide

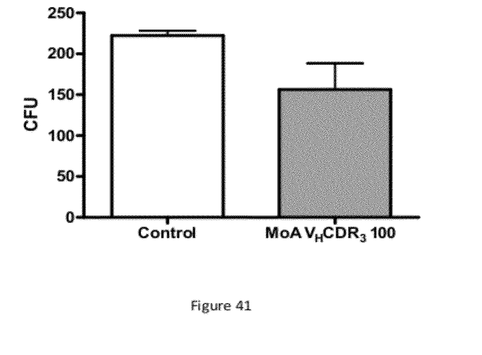

In vitro activity of MoA V_{H}CDR₃ against M. furfur 101 strain. The fungicidal activity of MoA V_{H}CDR₃ peptide against M. furfur has been evaluated by a conventional CFU assay. Cells of M. furfur 101 have been incubated at 30° C. for 6 hours in the presence of peptides at the concentration of 100 micrograms/ml, or in distilled water as control growth. After the incubation period, the cell suspensions have been plated on Sabouraud dextrose agar added with Tween 20 (1%), then incubated at 30° C. and observed for CFU enumeration after 72 hours. As disclosed in FIG. 41, MoA V_{H}CDR₃ showed fungicidal activity against M. furfur.

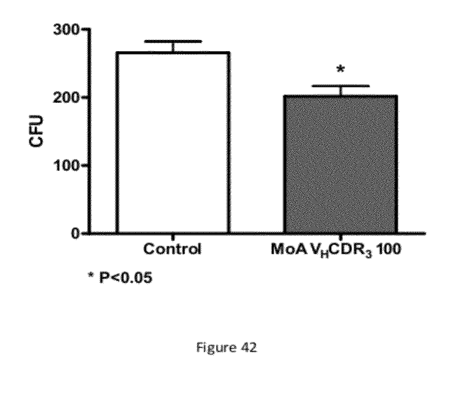

In vitro activity of MoA V_{H}CDR₃ against A. fumigatus 1163 strain. The fungicidal activity of H4L peptide against A. fumigatus has been evaluated by a CFU assay. Conidia of A. fumigatus 1163 have been incubated at 30° C. for 18 hours in the presence of MoA V_{H}CDR₃ at the concentration of 100 micrograms/ml, or in distilled water as control growth. After the incubation period, the cell suspensions have been plated on Sabouraud dextrose agar, then incubated at 30° C. and observed for CFU enumeration after 48 hours. As disclosed in FIG. 42, MoA V_{H}CDR₃ showed fungicidal activity against A. fumigatus (* P<0.05, treated versus untreated cells, t test).

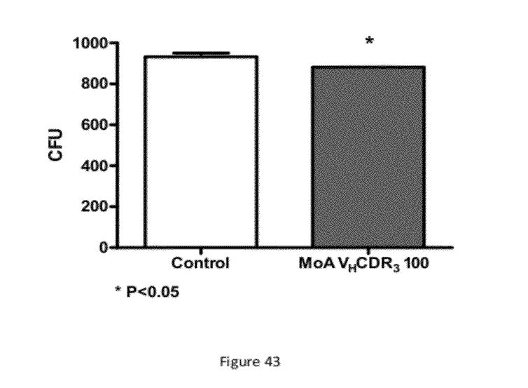

In vitro activity of MoA V_{H}CDR₃ against S. aureus 29213 strain. The microbicidal activity of MoA V_{H}CDR₃ peptide against S. aureus has been evaluated by a conventional CFU assay. Cells of S. aureus 29213 have been incubated at 37° C. for 5 hours in the presence of MoA V_{H}CDR₃ at the concentration of 100 micrograms/ml, or in distilled water as control growth. After the incubation period, the cell suspensions have been plated on Mueller Hinton agar, then incubated at 37° C. and observed for CFU enumeration after 24 hours. As disclosed in FIG. 43, MoA V_{H}CDR₃ has showed bactericidal activity against S. aureus (* P<0.05, treated versus untreated cells, t test).

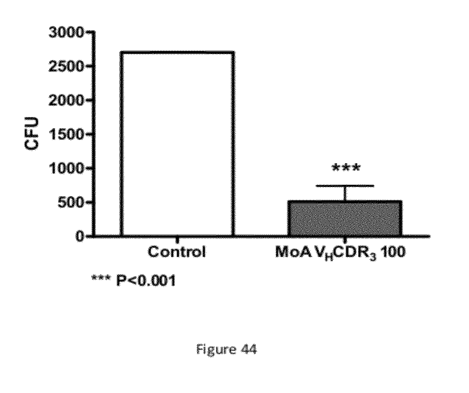

In vitro activity of MoA V_{H}CDR₃ against E. coli ATCC 25922 strain. The bactericidal activity of MoA V_{H}CDR₃ peptide against E. coli has been evaluated by a conventional CFU assay. Cells of E. coli ATCC 25922 have been incubated at 37° C. for 5 hours in the presence of MoA V_{H}CDR₃ at the concentration of 100 micrograms/ml, or in distilled water as control growth. After the incubation period, the cell suspensions have been plated on Mueller Hinton agar, then incubated at 37° C. and observed for CFU enumeration after 24 hours. As disclosed in FIG. 44, MoA V_{H}CDR₃ showed bactericidal activity against E. coli (*** P<0.001, treated versus untreated cells, t test).

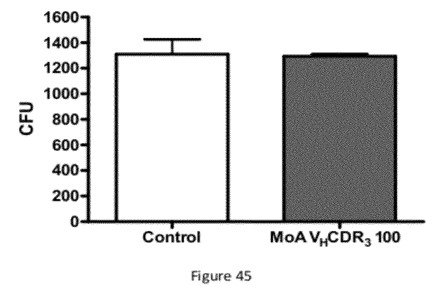

In vitro activity of MoA V_{H}CDR₃ against K. pneumoniae ATCC 700603 strain. The bactericidal activity of MoA V_{H}CDR₃ peptide against K. pneumoniae has been evaluated by a conventional CFU assay. Cells of K. pneumoniae ATCC 700603 have been incubated at 37° C. for 5 hours in the presence of MoA V_{H}CDR₃ at the concentration of 100 micrograms/ml, or in distilled water as control growth. After the incubation period, the cell suspensions have been plated on Mueller Hinton agar, then incubated at 37° C. and observed for CFU enumeration after 24 hours. As disclosed in FIG. 45, MoA V_{H}CDR₃ showed bactericidal activity against K. pneumoniae.

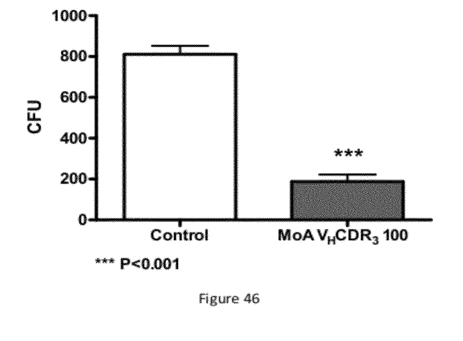

In vitro activity of MoA V_{H}CDR₃ against P. aeruginosa ATCC 25853 strain. The bactericidal activity of MoA V_{H}CDR₃ peptide against P. aeruginosa has been evaluated by a CFU assay. Cells of P. aeruginosa ATCC 25853 have been incubated at 37° C. for 5 hours in the presence of MoA V_{H}CDR₃ at the concentration of 100 micrograms/ml, or in distilled water as control growth. After the incubation period, the cell suspensions have been plated on Mueller Hinton agar, then incubated at 37° C. and observed for CFU enumeration after 24 hours. As disclosed in FIG. 46, MoA V_{H}CDR₃ showed bactericidal activity against P. aeruginosa (*** P<0.001, treated versus untreated cells, t test).

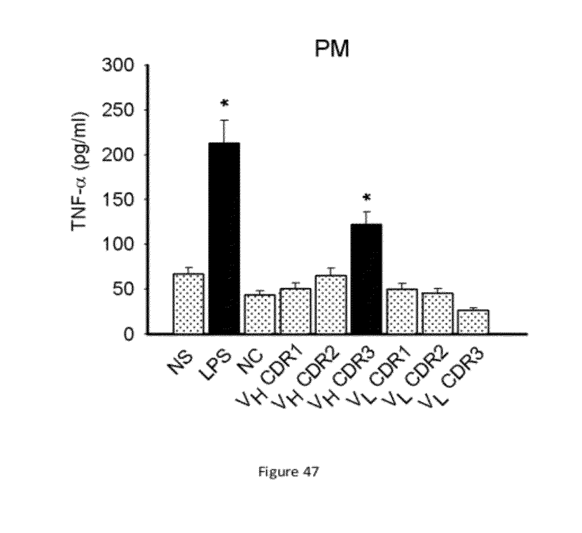

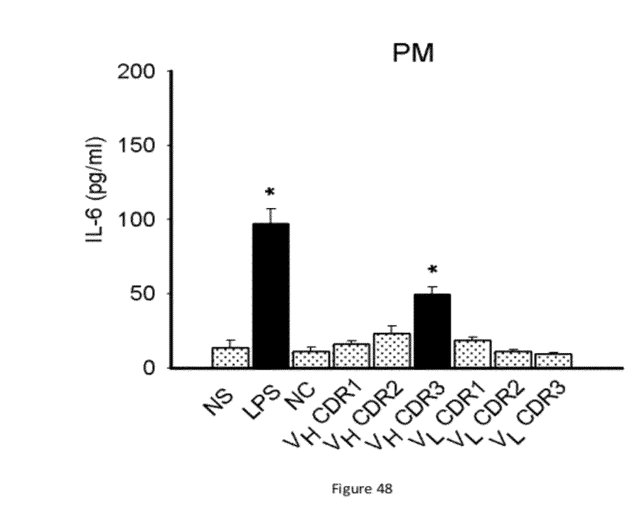

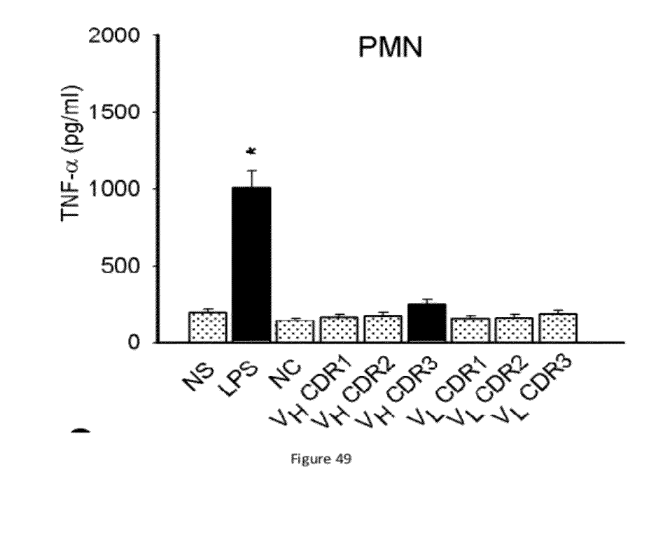

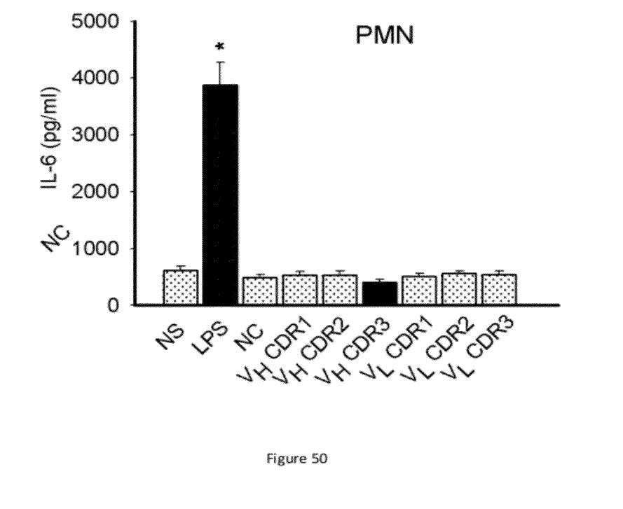

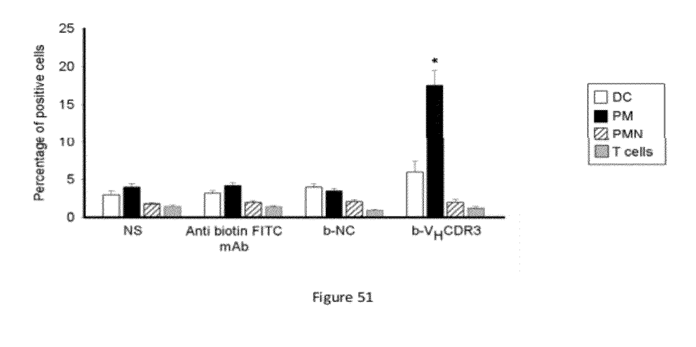

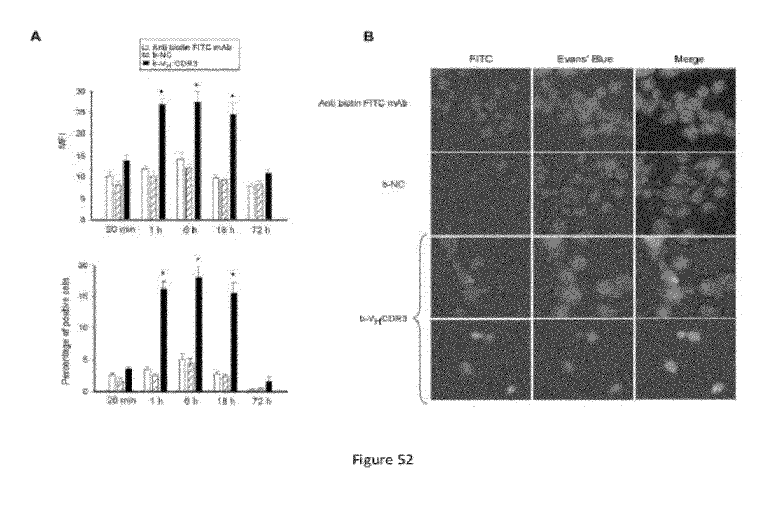

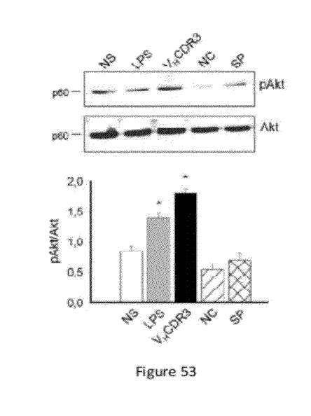

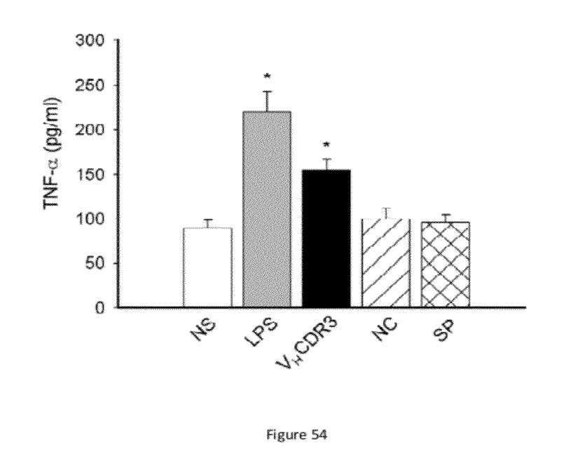

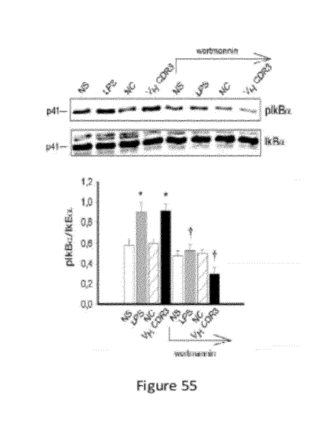

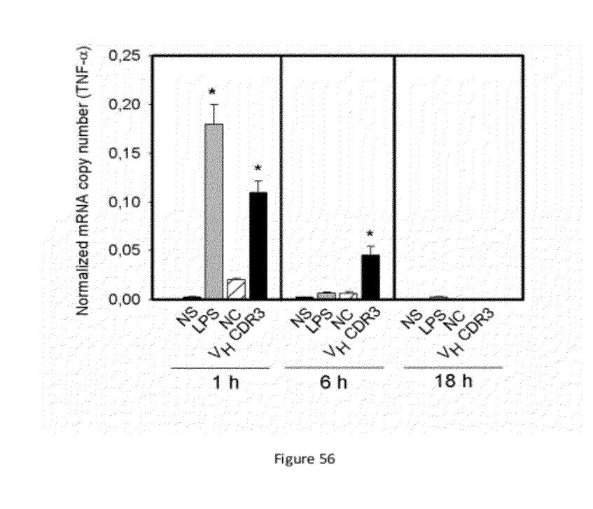

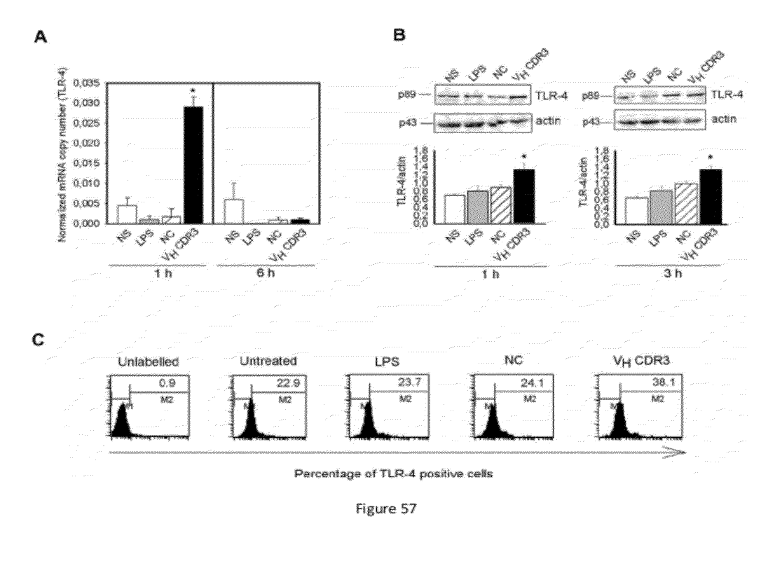

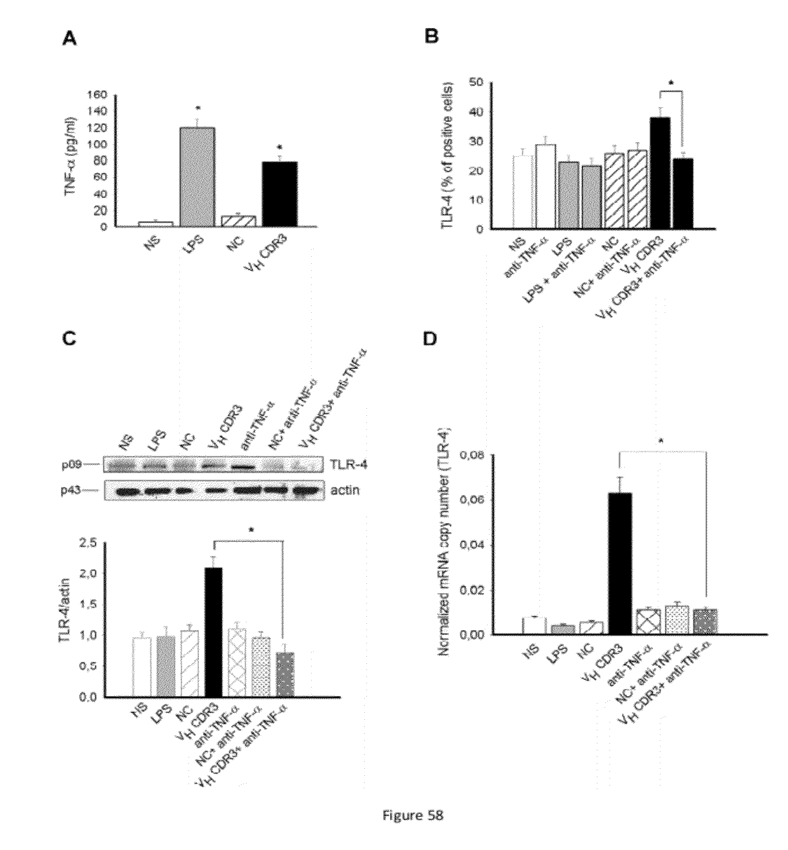

In vitro immunomodulatory activity of MoA V_{H}CDR₃ on murine immune cells. FIGS. 47-51 are graphs showing TNF-α and IL-6 production by PM and PMN stimulated with MoA CDRs and mouse MoA V_{H}CDR₃ uptake by different cell populations. Mab MoA CDR sequences experiments with PM are shown in FIGS. 47 and 48. Mab MoA CDR sequences experiments with PMN are shown in FIGS. 49 and 50. Cells were cultured in the presence or absence (NS) of human and/or mouse CDRs, lipopolisaccharide (LPS), or negative control peptide (NC) (all 10 micrograms/ml) for 18 hr. Both PM and PMN cell populations were 5×10⁶/ml. After incubation, TNF-α and IL-6 levels were evaluated in culture supernatants by specific ELISA assays (* P<0.05 treated vs untreated cells, n=7). In FIG. 51, dendritic cells (DC), PM, PMN, and T cells (all 1×10⁶/ml) were incubated for 1 hr in the presence or absence (NS) of biotinylated (b)-MoA V_{H}CDR₃ or b-NC (both 10 micrograms/ml). After incubation, permeabilized cells were reacted with FITC-labelled mAb to biotin (b) and analyzed by FACScan flow cytometry. Data are reported as the percentage of positive cells *, P<0.05 (b-V_{H}CDR₃ treated vs untreated cells, n=5). Error bars, s.e.m. FIGS. 52A and B show graphs and colored cell staining micrograph of kinetic of V_{H}CDR₃ uptake by PM. PM (1×10⁶/ml) were incubated for different times with b-V_{H}CDR₃ or b-NC (all 10 micrograms/ml). After incubation, permeabilized cells were reacted with FITC-labelled mAb to biotin and analyzed by FACScan flow cytometry. Data are reported as the mean fluorescence intensity (MFI) and percentage of positive cells (FIG. 52A) (* P<0.05 b-V_{H}CDR₃-treated vs untreated cells, n=7). Error bars, s.e.m. In selected experiments, cells were incubated for 1 hr as above described, reacted with FITC-labelled mAb to biotin in the presence of Evans' Blue as a counter stain, and subsequently examined under fluorescent light microscopy (FIG. 52B). Note the green fluorescence of b-V_{H}CDR₃ treated cells. Original magnification 20×. In FIGS. 53 and 54 is shown the phospho-Akt activation and TNF-α production in PM stimulated with MoA V_{H}CDR₃. PM (3×10⁶/ml) were stimulated for 1 hr in the presence or absence (NS) of MoA V_{H}CDR₃, LPS, an irrelevant control peptide (NC) or a scrambled peptide (SP) (all 10 micrograms/ml). After incubation, cell lysates were subjected to Western blotting. Membranes were incubated with Abs to pAkt and Akt; pAkt was normalized against Akt (FIG. 53)*, P<0.05 (treated vs untreated cells, n=5). FIG. 54 shows the production of TNF-alpha in murine macrophages stimulated with MoA V_{H}CDR₃. PM (5×10⁶/ml) were stimulated for 18 hr as above described. After incubation, TNF-α level was evaluated in culture supernatants by specific ELISA assays. (FIG. 54)*, P<0.05 (treated vs untreated cells, n=5). In FIGS. 55 and 56 is shown the phospho-IkBα activation and TNF-α gene expression in PM stimulated with MoA V_{H}CDR₃. PM (3×10⁶/ml) were stimulated for 1 hr in the presence or absence (NS) of wortmannin (4 nM), MoA V_{H}CDR₃, LPS or NC (all 10 micrograms/ml). After incubation, cell lysates were subjected to Western blotting. Membranes were incubated with Abs to pIkBα and IkBα; pIkBα was normalized against IkBα. (FIG. 55)*, P<0.05 (treated versus untreated cells, n=5); †, P<0.05 (wortmannin-treated vs wortmannin-untreated cells, n=5). For testing the expression level of TNF-α gene, PM (1×10⁶/ml) were cultured for 1, 6 and 18 hr as above described. After incubation, total RNA was isolated and analyzed for mRNA expression with RT-PCR (FIG. 56). Transcript copy numbers were determined by qPCR using cDNA as a template. Copy numbers were normalized against the copy number of the GADPH gene (B). (* P<0.05 (treated vs untreated cells, n=5). Error bars, s.e.m. In FIG. 57 is shown the expression of TLR-4 in PM stimulated with MoA V_{H}CDR₃. PM (1×10⁶/ml) were cultured for 1 and 6 hr in the presence or absence (NS) of MoA V_{H}CDR₃, LPS or NC (all 10 micrograms/ml). After incubation, total RNA was isolated and analyzed for mRNA expression with RT-PCR. Transcript copy numbers were determined by qPCR using cDNA as a template. Copy numbers were normalized against the copy number of the GADPH gene (FIG. 57 A) (* P<0.05 (MoA V_{H}CDR₃ treated vs untreated cells, n=5). PM (3×10⁶/ml) were cultured for 1 and 3 hr as above described. After incubation, cell lysates were subjected to Western blotting. Membranes were incubated with Abs to TLR-4 and actin. TLR-4 production was normalized against actin (FIG. 57 B) (*, P<0.05 MoA V_{H}CDR₃-treated vs untreated cells, n=5). Error bars, s.e.m. PM (1×10⁶/ml) were incubated for 1 hr as above described. After incubation, permeabilized cells were reacted with RPE-labelled mAb to TLR-4 and analyzed by FACScan flow cytometry. Values represent the percentage of positive cells (FIG. 57 C). TNF-α induced TLR-4 expression in PM stimulated with MoA V_{H}CDR₃ (FIG. 58). PM (5×10⁶/ml) were cultured for 1 hr in the presence or absence (NS) of MoA V_{H}CDR₃, LPS or NC (all 10 micrograms/ml). After incubation, TNF-α level was evaluated in culture supernatants by specific ELISA assay (FIG. 58A) (* P<0.05 treated vs untreated cells, n=7). PM (1×10⁶/ml) were cultured for 1 hr with V_{H}CDR₃, LPS or NC (all 10 micrograms/ml), in the presence or absence (NS) of mAb to TNF-α (0.5 micrograms/ml). After incubation, permeabilized cells were reacted with RPE-labelled mAb to TLR-4 and analyzed by FACScan flow cytometry. Values represent the percentage of positive cells (FIG. 58B) (* P<0.05 V_{H}CDR₃ plus mAb to TNF-α treated vs MoA V_{H}CDR₃ treated cells, n=7). PM (3×10⁶/ml) were cultured for 1 hr as above described. After incubation, cell lysates were subjected to Western blotting. Membranes were incubated with Abs to TLR-4 and actin. TLR-4 production was normalized against actin (FIG. 58C) (* P<0.05 V_{H}CDR₃ plus mAb to TNF-α treated vs MoA V_{H}CDR₃ treated cells, n=5). For testing the expression level of TLR-4 gene, PM (1×10⁶/ml) were cultured for 1 hr as above described. After incubation, total RNA was isolated and analyzed for mRNA expression with RT-PCR. Transcript copy numbers were determined by qPCR using cDNA as a template. Copy numbers were normalized against the copy number of the GADPH gene (FIG. 58D) (* P<0.05 MoA V_{H}CDR₃ plus mAb to TNF-α treated vs V_{H}CDR₃ treated cells, n=5). Error bars, s.e.m.

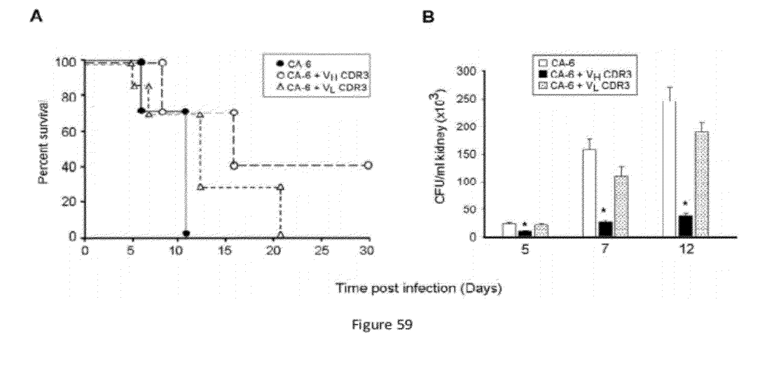

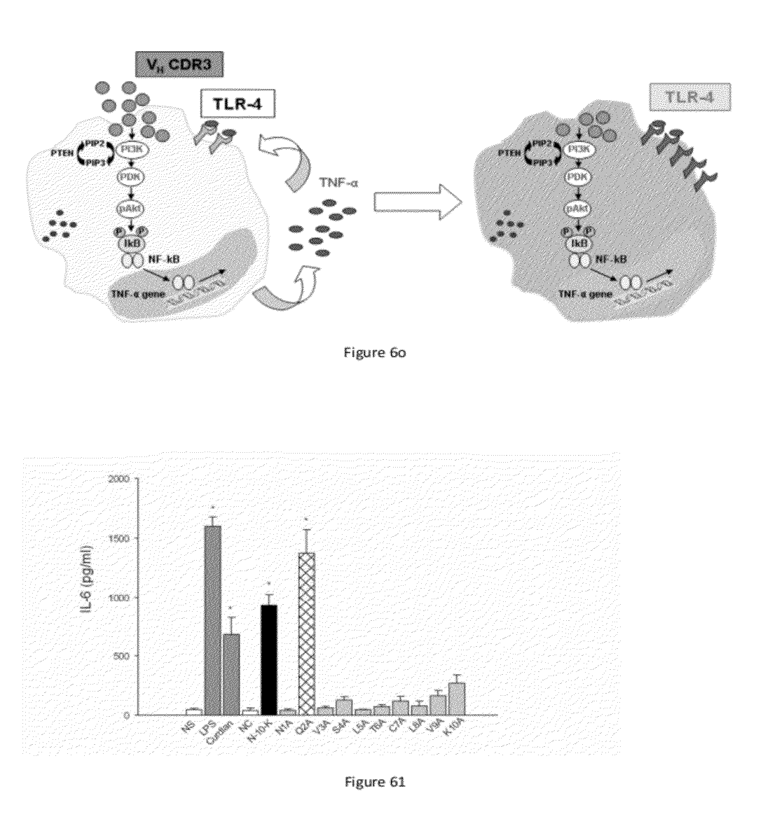

Given that V_{H}CDR₃ is able to induce a state of activation in PM, we tested whether this condition could influence the course of infection in a mouse experimental model of systemic candidiasis, despite the proven non-candidacidal properties of the peptide. Mice were infected intravenously with the opportunistic fungus C. albicans and treated with mouse V_{H}CDR₃ or V_{L}CDR₃ (used as a negative control) intraperitoneally 4 hr before (200 micrograms), and 1 (100 micrograms) and 2 (100 micrograms) days after infection. Animal survival and fungal burden in kidneys were evaluated in different groups of mice. Percent survival and determination of fungal clearance from kidneys of Balb/c mice challenged with C. albicans (CA-6) and treated with MoA V_{H}CDR₃ or MoA V_{L}CDR₃ are disclosed in FIG. 59. Percent survival of infected mice was evaluated according to Mantel-Cox Log rank test and the difference among experimental groups resulted significant (* P<0.05, n=7) (FIG. 59A). CFU recovery from the kidneys of mice was determined 5, 7 and 12 days after fungal infection (FIG. 59B) (* P<0.05 MoA V_{H}CDR₃ treated versus untreated mice, n=7). Error bars, s.e.m. Results, reported in FIG. 59A, showed a significant increase in survival for infected mice treated with MoA V_{H}CDR₃ as compared to infected mice untreated or treated with the non immunomodulatory MoA V_{L}CDR₃. In the same experimental conditions, CFU recovery from kidneys showed an impressive decrease when mice infected with C. albicans were treated with MoA V_{H}CDR₃ as compared to negative control mice (FIG. 59B). FIG. 60 is a pictoral diagram delineating the mechanism of TLR-4 upregulation induced in PM by MoA V_{H}CDR₃. As a proof of concept, here we demonstrate that a synthetic peptide with sequences identical to MoA V_{H}CDR₃ of a mouse IgM mAb specific for difucosyl human blood group A substance (MoA) could display a potent immunomodulatory activity thus exerting a therapeutic effect against systemic candidiasis without possessing direct candidacidal properties. Significantly, the aminoacidic residue at position 5 (N) proved to be functionally critical for the immunostimulatory properties of MoA V_{H}CDR₃, as its substitution with alanine resulted in a loss of TNF-α production capacity. One possible scenario suggested by these data is that selected short sequences representative of the CDRs of Abs could be strongly involved in inflammatory responses and, as a consequence, in chronic inflammatory processes. MoA V_{H}CDR₃ peptide is able, indeed, to stimulate PM to produce TNF-α, and this could be instrumental in inducing inflammation. As a matter of fact, TNF-α is considered a classical cytokine of chronic inflammatory disease.

PM perform a central task in both the innate and adaptive immune systems. The life and function of these cells are characterized by significant functional versatility. PM ingest foreign materials, present Ags to T lymphocytes in association with the MHC, and can kill microbes and tumor cells upon activation by cytokines and/or T cells. In addition, they eliminate damaged or apoptotic cells. Conversely, PM can also release copious amounts of toxic metabolites that can promote tissue damage during antimicrobial defense responses.

Our evidence reports that PM very rapidly take up the MoA V_{H}CDR₃ peptide, and 18 hr post treatment this peptide is still associated to the cells. It is possible that MoA V_{H}CDR₃ could be continuously internalized and degraded within 18 hr; alternatively, the peptide could be retained by cells for 18 hr and subsequently degraded or expelled.

PI3K has been linked to an extraordinarily diverse group of cellular functions, including cell growth, proliferation, differentiation, motility, survival and intracellular trafficking Many of these functions relate to the ability of PI3K to activate Akt. The interaction of MoA V_{H}CDR₃ with PM induces Akt activation that finally leads to phosphorylation of IkBalpha with consequent translocation of NFkB into the nucleus. These molecular events are responsible for cellular activation and subsequent transcription of proinflammatory cytokine genes such as TNF-α. Indeed, this pathway of activation is also confirmed by the inhibition of TNF-α production after blocking the specific Akt signalling pathway. Similarly, involvement of p38 MAPK activation was detected using a specific inhibitor of this pathway. As a matter of fact, TNF-α mRNA was detected 1 hr post stimulation with MoA V_{H}CDR₃, suggesting that the signal transduction pathway from Akt leads to cytokine gene expression, as depicted in FIG. 60.

Given that TNF-α is believed to be a positive regulator of TLR-4 expression, and that the ability of cells to respond to several microbial motifs depends on TLR-4 expression, we found that, in our experimental system, MoA V_{H}CDR₃ up-regulates TLR-4. The stimulation of TLR-4 leads to cellular activation, and this effect could reinforce the capacity of the peptide to induce inflammatory responses. Moreover, when considering that TLR-4 up-regulation is completely blocked by neutralizing TNF-α, one could posit that the over expression of TLR-4 is secondary and dependent on TNF-α production.

In relation to its protective response, there are convincing arguments pointing to the key role of TLR-4 in microbial antigen recognition. In particular, the antigenic structures of the opportunistic fungus C. albicans are recognized by TLR-4. In our experimental system, a significant increase in survival and a drastic decrease in fungal growth in the kidney, the target organ for C. albicans, was surprising, given that MoA V_{H}CDR₃ is ineffective against C. albicans cells in vitro. A possible explanation for this could be that natural immune cells are activated by MoA V_{H}CDR₃ treatment and more prone to ingest and kill C. albicans. Additionally, increased TLR-4 expression on PM could facilitate C. albicans recognition with consequent more prompt and efficient immune response. The rapid clearance of C. albicans observed in vivo is particularly relevant, given that MoA V_{H}CDR₃ does not show any direct candidacidal activity. A simple peptide derived from a mAb specific for difucosyl human blood group A substance, is endowed with potent immunoregulatory effects that are intrinsically able to control the course of a microbial infection.

Whether a proteolytic release of modulatory fragments may physiologically occur beyond the half life of Igs is an intriguing hypothesis that would account for the apparent redundancy in their production. Nature may have provided extrinsic activities to peptides integrated in evolutionary molecules such as Abs in a way reminiscent of human cationic peptides that play an innate immune regulatory role in host defense. Overall these findings suggest that Ab-derived peptides can act likewise effectors of the innate immune response opening a new scenario about their interplay with the cellular immune response.

### 5. Experiment V

### Data for NK10 Alanine Substitution Variants

In Vitro Comparative Activity of N10K and N10K Alanine-Substituted Derivatives (asd) Against Candida albicans SC5314 Strain

Derivatives of the decapeptide N10K were obtained by alanine scanning in order to improve its candidacidal activity and identify the functional contribution of each residue. The alanine-substituted derivatives (asd), defined according to the position held by the alanine-substituted aminoacid, were tested against C. albicans by a conventional colony forming unit (CFU) as previously described (Polonelli et al., 2003). Briefly, cells of C. albicans SC5314 were incubated at 37° C. for 6 hours in the presence of N10K or N10K asd at different scalar concentrations or distilled water as control. After incubation, cell suspensions were plated on Sabouraud dextrose agar and incubated at 30° C. for 48 hours when CFU were counted. Based on several independent replications, the EC₅₀ value of each peptide was calculated by nonlinear regression analysis using Graph Pad Prism 4.01 software, San Diego, Calif., USA. As disclosed in Table IX, N10K asd showed differential candidacidal activity in vitro against cells of C. albicans SC5314 strain in comparison to N10K. We note that with respect to the present experiments in this example NK10 and its derivatives show, generally, a higher activity level than the activity levels observed in NK10 peptide experiments presented in Example I. This is because the peptides, purified for use in the present example, were processed to a greater extent than for the NK10 in the experiments discussed in Example I suggesting that per unit used, the concentration of active peptide in the experiments of this example is greater per unit volume.

TABLE IX

| | | | | 95% | | EC₅₀asd/ |
|---|---|---|---|---|---|---|
| Peptide | sequence | MW | EC₅₀ mol/L | confidence intervals | | EC₅₀N10K |
| N10K | NQVSLTCLVK | 1104.33 | 1.004 × 10⁻⁶ | 0.947 × 10⁻⁶ | 1.065 × 10⁻⁶ | 1 |
| SEQ ID NO: 1 | | | | | | |
| asd N1A N10K, | AQVSLTCLVK | 1061.30 | 5.871 × 10⁻⁷ | 5.862 × 10⁻⁷ | 5.881 × 10⁻⁷ | 0.58 |
| Seq Id. No. 14 | | | | | | |
| asd Q2A N10K | NAVSLTCLVK | 1047.28 | 2.305 × 10⁻⁶ | 2.260 × 10⁻⁶ | 2.350 × 10⁻⁶ | 2.30 |
| Seq Id. No. 15 | | | | | | |
| asd V3A N10K | NQASLTCLVK | 1076.28 | 5.929 × 10⁻⁶ | 5.064 × 10⁻⁶ | 6.941 × 10⁻⁶ | 5.91 |
| Seq Id. No. 16 | | | | | | |
| asd S4A N10K | NQVALTCLVK | 1088.33 | 2.391 × 10⁻⁶ | 2.076 × 10⁻⁶ | 2.755 × 10⁻⁶ | 2.38 |
| Seq Id. No. 17 | | | | | | |
| asd L5A N10K | NQVSATCLVK | 1062.25 | 8.847 × 10⁻⁷ | 8.415 × 10⁻⁷ | 9.311 × 10⁻⁷ | 0.88 |
| Seq Id. No. 18 | | | | | | |
| asd T6A N10K | NQVSLACLVK | 1074.30 | 4.878 × 10⁻⁶ | 4.364 × 10⁻⁶ | 5.453 × 10⁻⁶ | 4.86 |
| Seq Id. No. 19 | | | | | | |
| asd C7A N10K | NQVSLTALVK | 1072.27 | 5.493 × 10⁻⁵ | 2.596 × 10⁻⁶ | 1.162 × 10⁻³ | 54.71 |
| Seq Id. No. 20 | | | | | | |
| asd L8A N10K | NQVSLTCAVK | 1062.25 | 7.435 × 10⁻⁷ | 7.322 × 10⁻⁷ | 7.549 × 10⁻⁷ | 0.74 |
| Seq Id. No. 21 | | | | | | |
| asd V9A N10K | NQVSLTCLAK | 1076.28 | 1.776 × 10⁻⁶ | 1.742 × 10⁻⁶ | 1.810 × 10⁻⁶ | 1.77 |
| Seq Id. No. 22 | | | | | | |
| asd K10A N10K | NQVSLTCLVA | 1047.23 | * | | | |
| Seq Id. No. 23 | | | | | | |

* EC₅₀ was not determined, asd K10A showed a percentual inhibition of C. albicans growth equal to 69.04 at the concentration of 250 micrograms/ml

Thus, as indicated by the data presented in Table IX, alanine substitutions of NK10 peptide sequence show activity against yeast type fungus.

In Vitro Immunomodulatory Activity of N10K and N10K asd on Human Immune Cells.

The same battery of N10K asd was tested for their immunomodulatory activity on human monocytes or polymorphonucleated leukocyte PMN as previously described (Gabrielli et al., 2009). In particular, monocytes or PMN (both 10×10⁶/ml) were incubated in RPMI 1640 plus 10% FCS for 18 hr or 6 hr respectively in the presence or in the absence (NS) of LPS as a positive control, an inactive peptide (MSTAVSKCAT, Seq. ID. No. 26) as a negative control (NC) and N10K and N10k asd peptides (all 10 μg/ml). After incubation culture supernatants were collected and tested for cytokines production by specific ELISA. As disclosed in FIG. 61, N10K and N10K asd differentially stimulated the production of I1-6 (* P<0.05 treated vs untreated). In particular, the substitution of glutamine with alanine (peptide Q2A) resulted in a strong enhancement of the induction of IL-6 production by human monocytes.

Specifically, FIG. 61 shows IL-6 production. Monocytes (20×10⁶/ml were incubated for 18 hr at 37° C. plus 5% CO₂ in the presence or absence (NS) of LPS (10 micrograms/ml), Curdlan (25 micrograms/ml), N10K, and N10K alanine-substituted derivatives (all 10 micrograms/ml). NC, negative control. After incubation, the supernatants were collected and tested for IL-6 production by specific ELISA. *P<0.05 treated versus untreated.

### 6. Experiment VI

### Data for T11F Alanine Substitution Variants

The microbicidal activity of T-11-F peptide alanine scanning derivatives (Seq. Id. Nos. 27 to 37) were evaluated by a conventional colony forming unit (CFU) assay. Cells of C. albicans SC5314 were incubated at 37° C. for 6 hours in the presence of peptides at the concentration of 5, 3 or 2 micrograms/ml, or in distilled water as control growth. After the incubation period, the cell suspensions were plated on Sabouraud dextrose agar, then incubated at 30° C. and observed for CFU enumeration after 48 hours.

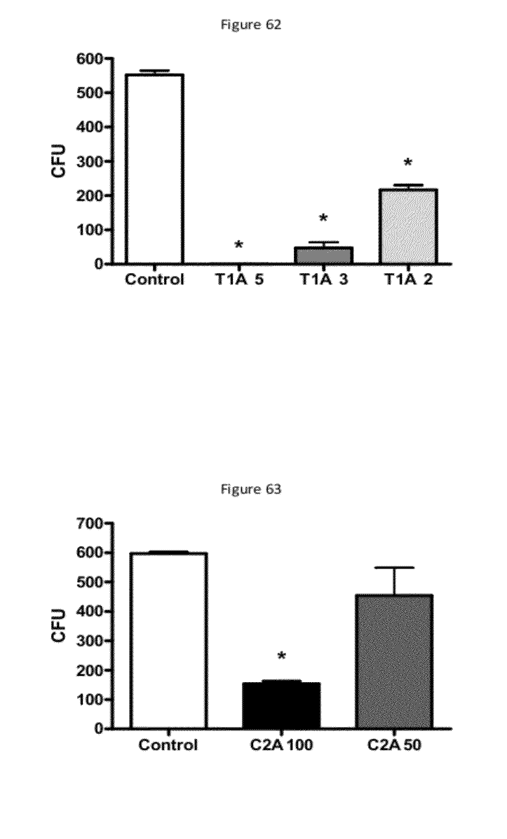

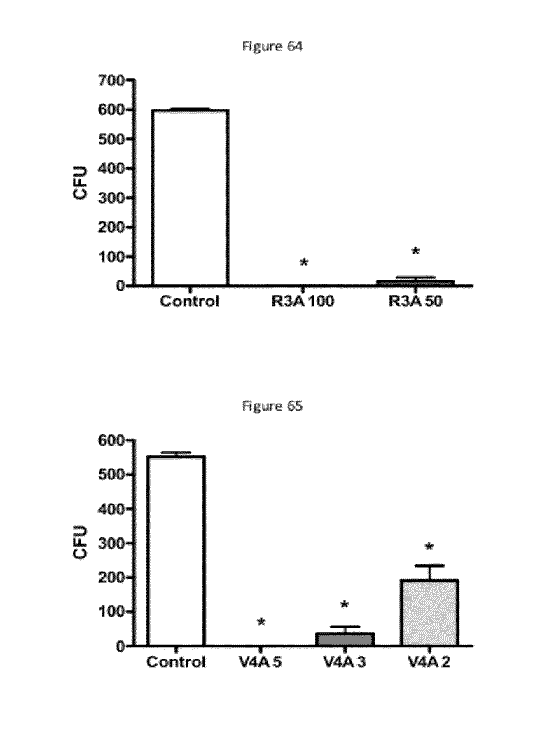

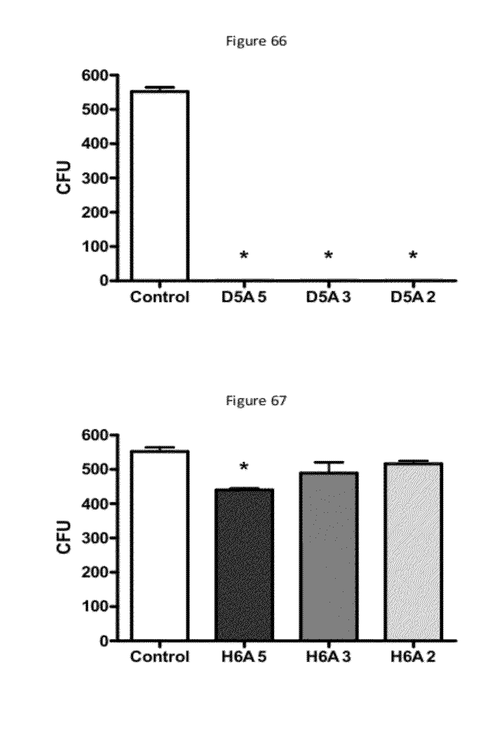

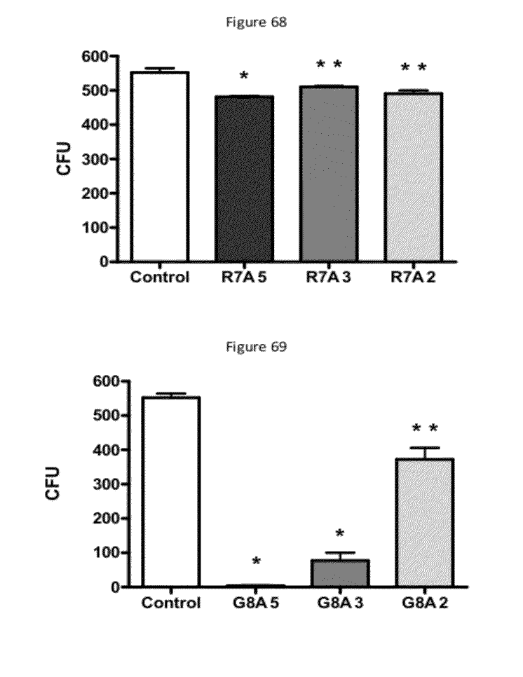

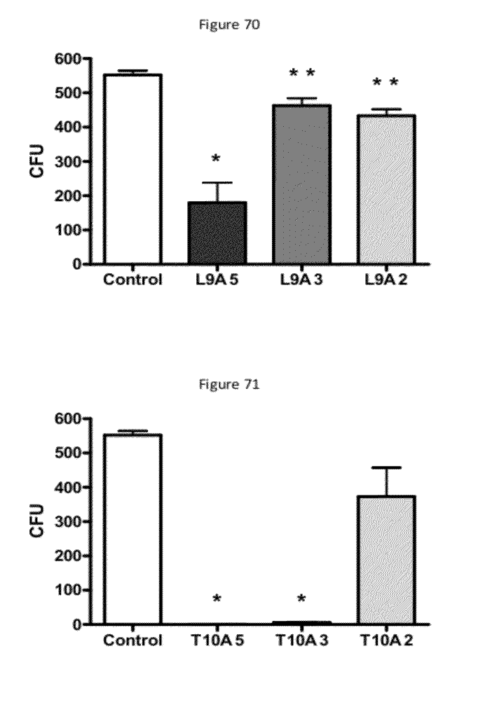

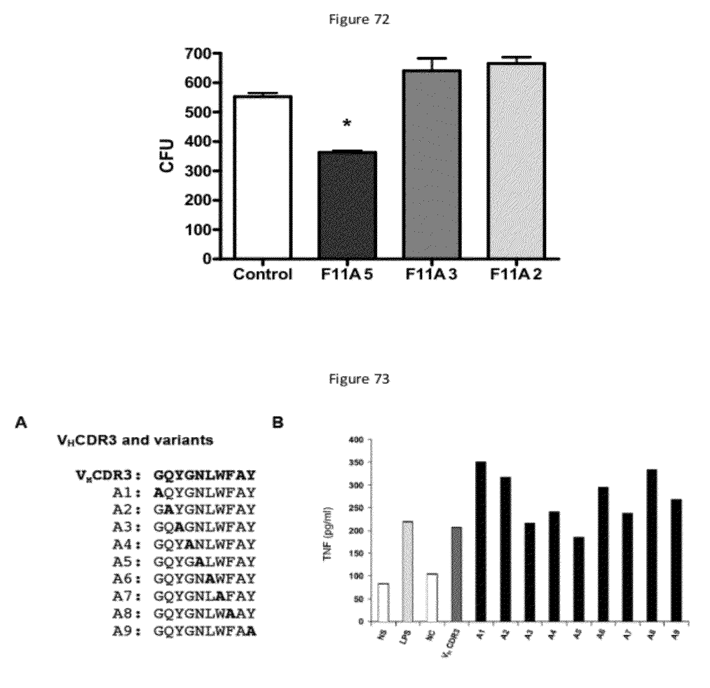

As shown in FIGS. 62 to 72 graphs are provided which depict the activity level of T11F alanine scanning derivatives. The graphs show that each of the peptides are active to one degree or another.

### 7. Experiment VII

### Data for N10K Alanine/Serine Double Substitution Variant

In vitro microbicidal activity of N10K derivative with 2 amino acid substitutions, or 80% sequence identity with native N10K, namely peptide Seq Id. No. 25 was tested against Candida albicans SC5314 as determined by Colony Forming Unit assay. As shown in Table X, candidacidal activity is present in a peptide derived from N10K with two amino acids substitutions.

TABLE X

| Peptide | Sequence | Mol Wt. | EC₅₀ mol/L | 95% confidence intervals | |
|---|---|---|---|---|---|
| L5A, L8S N10K, | NQVSATCSVK | 1036.17 | 4.321 × 10⁻⁵ | 4.240 × 10⁻⁶ | 4.403 × 10⁻⁴ |
| (SEQ ID NO: 25) | | | | | |

### 8. Experiment VIII

### Data for MoA V_{H}CDR₃ Alanine Substitutions on Immune Stimulatory Activity

FIG. 73 shows TNF-α inducing activity of peptides derived from VHCDR3 by single amino acid substitution. Part A) depicts the battery of single amino acid alanine-scanning substitutions of the immunomodulatory peptide VHCDR3. Part B) shows TNF-α production by mouse peritoneal macrophages treated with the VHCDR3 variants in A. All but two of the alanine-containing mutant peptides showed increased TNF-α induction over VHCDR3. Four peptides (A1,2,6,8) had increases of approximately 50% or more; while 2 peptides (A3,5) had TNF-α-inducing activity that were either unchanged or slightly reduced. NS: no peptide added (not stimulated); NC: negative control peptide; LPS: lipopolysaccharide (positive control).

### Peptide Applications

In further embodiments, the invention peptides can be used in various methodologies for treating invasive bacterial, fungal (yeast and mold), and viral infections as well as be useful for treating cancer disorders or other therapies that benefit from immune modulation such as modulations that otherwise affect such factors as cytokines. In preferred embodiments, it is contemplated that such treatments can include both topical and systemic applications as in our data the peptides prove to have exceptionally low toxicity. The peptides of the invention can be administered to a mammal infected with a fungus, topically or systemically, such as in the case of an infection with Aspergillus sp. a yeast such as in the case of vulvovaginal candidiasis caused by C. albicans, or a mammal infected with a bacterium such as E. coli. Other invasive organisms contemplated for treatment in mammals by administration of the peptide of the invention topically or systemically include Mycobacterium tuberculosis, Cryptococcus spp., Fusarium spp., Scedosporium spp., Histoplasma capsulatum, Blastomyces dermatitidis, Zygomycetes and dematiaceous fungi.

### Treatment Applications

Topical administration can be cream or ointment based, the respective formulation comprising active and inert materials as are commonly known for such topical treatments and at concentrations as proven useful in mammalian models. Systemic administration can be by injection wherein the formulary of the injectate comprises salts and solutions well known for administering peptides in such fashion. For example, concentration of the invention peptides identified by Seq. Id Nos. 1-13 in either topical or systemic formulations can comprise concentration ranges of between 2 and 100 micrograms/ml, more typically any of 2-5 micrograms/ml, 3-5 micrograms/ml, 5-10 micrograms/ml, 5-20 micrograms/ml, 30-50 micrograms/ml, 40-50 micrograms/ml, 50-100 micrograms/ml. In some applications, as little as 1-2 micrograms/ml is effective. Other concentrations include 2, 3, 5, 10, 20, 30, 40, 50, 60, 80, and 100 micrograms/ml.

Additional, treatment regimens include length of time periods for which treatment by topical or systemic application should be made. In preferred embodiments treatment regimens contemplate delivery over at least 4 days. In a particularly preferred embodiment, application or otherwise delivery of the antimicrobial (antibacterial and antifungal), antiviral, or immunomodulatory polypeptide is made between one and four days and alternatively every four days or (24 hr period), or alternatively, continuously over a period of at least 4 days with application ranging from once, twice, three or even four times per day and depending upon the ultimate dosage concentration used. Dosing can be carried on for periods of up to one month or more.

### Consensus Motifs

In yet additional embodiments of the current invention, the naturally occurring Ig C region and CDR region peptide, as well as their alanine, serine, or otherwise 80 to 90% identity substituted derivatives, possess a similar primary structure given the evolutionarily accepted equivalent amino acid substitutions as one of ordinary skill in the art will understand as depicted in Table IV. Thus, at the base level of characterization, the amino acid sequences of the invention possess unity of invention based on the similar charge configurations of the various possible R groups positioned in sequence to one another as illuminated through the consensus formula XZ*1X1Z*11XZ as shown in Table IV. The varied amino acid sequence type motif of the present formula provides support for the conception that the active motif comprises between one and three large aromatic, or neucleophilic, or otherwise charged (basic or acid) amino acid groups (can be paired amino acids) that are spaced from one another (pairs, if present, are spaced from other pairs) by between two to three amino acids that are themselves either small, aliphatic, or hydrophopic (non polar or small polar R groups). Each of X, *, Z, and 1 represent amino acids of varying types having structural and/or evolutionary similarity or interchangeability. For example, amino acid “Z” is a structural amino acid. Amino acid Z, and the combination Z*, (where “*” represents a second structural amino acid, if present, making a double amino acid structural motif “Z*”), are generally large charged (basic or acidic), aromatic or bulky amino acids, or that possess cross-linking capability, such a Cysteine, in the polypeptide number 2-3, 6-7-8, and 10-11^{th} positions for 10 or 11 amino acid polypeptides. Thus depending upon the total length of the active polypeptide of the invention the position number of the type of amino acid can vary within discernable limits and still remain within the overriding formula disclosed above. Specifically, Z and * amino acids are also defined as any of Histidine (H), Cysteine (C), Glutamine (Q), Phenylalanine (F), Tryptophan (W), Tyrosine (Y), Lysine (K), Glutamic acid (E), and Arginine (R). With respect to amino acid “1” in polypeptide positions number 3-4, 5-6, 8-9, the amino acids in these positions are hydrophobic nonpolar and generally minimal sized R groups. Specifically, amino acids in “1” position can be Glycine (G), Valine (V), Leucine (L), Isoleucine (I), Methionine (M), and Alanine (A). Amino acids of the “X” type are generally small polar oxygen containing R group in the 4-5 and 9-10 amino acid positions. Amino acids of the X type can be Threonine (T), Serine (S), Asparagine (N), and Aspartic acid (D). We further note that the 1-X amino acid combinations can be mixed in sequence position. The consensus 1 formula may also be expressed in the alternative as consensus sequences 2 or 3 wherein the amino acid choice “*” is represented as “B” and can be cystine (C), Glutamine (Q), Glutamic acid (E), and Arginine (R), Tyrosine (Y), and Phenylalanine (F), the consensus 2 providing a listing of those amino acids interchangeable for each position, and consensus formula 3 providing a further derivation wherein generally hydrophobic aliphatic and small polar uncharged R group amino acids, labeled “Hyd” which can be any of amino acids Glycine (G), Valine (V) Leucine (L), Isoleucine (I), Methionine (M), and Alanine (A), Serine (S), Threonine (T), Asparagine (N), and Aspartic acid (D). A fourth consensus alignment motif is provided in the formula “Z-B-Hyd-Hyd” which can be active in singular and multiple repeat motifs as in T11F sequence motifs CRVD (Seq. Id. No. 38) and HRGL (Seq. Id. No. 39), or sequence H4L and its “HEAL” (Seq. Id. No. 3) amino acid motif, or N10K sequence motif “TCLV” (Seq. Id. No. 40), or MoA V_{H}CDR₃ sequence motif “QYGN” (Seq. Id. No. 41). Still further, another consensus motif is discernable in consensus 5 recognizing a motif of six amino acids wherein the terminal 5′ and 3′ amino acids are of the “Hyd” type while the inner amino acids possess the motif “Hyd-X-Z-B-Hyd-Hyd” as found in T11F (VDHRGL) (Seq. Id. No. 42), N10K (VSLTCL) (Seq. Id. No. 43), and MoA V_{H}CDR₃ “GNLWFA” (Seq. Id. No. 44). Further still a sixth consensus motive (consensus 6) is discernable in that a combination of consensus 4 and 5 but further defined where spacing allows for a “1” amino acid type present with a Histidine in “Z” position. Although each of the six formula motifs are discernable alone, they further in combination with one another provide for unity of invention with one another.

TABLE IV

| Peptide ID | Sequence ID | Peptide sequence | | | | | | | | | | | |
|---|---|---|---|---|---|---|---|---|---|---|---|---|---|
| Consensus 1 | | X | Z | * | 1 | X | 1 | Z | * | 1 | 1 | X | Z |
| Consensus 2 | | [TNG] | [CQ] | B | [VG] | [DSN] | L | [HTW] | B | [GLA] | [LV] | [TK] | [FY] |
| Consensus 3 | | [TNG] | [CQ] | B | Hyd | [DSN] | Hyd | [HTW] | B | Hyd | Hyd | [TK] | [FY] |
| Consensus 4 | | | Z | B | Hyd | Hyd | | Z | B | Hyd | Hyd | | |
| Consensus 5 | | | | | Hyd | X | | Z | B | Hyd | Hyd | | |
| Consensus 6 | | | | | Hyd | X | | L/H | B | Hyd | Hyd | | |
| T11F | 2 | T | C | R | V | D | | H | R | G | L | T | F |
| H4L | 3 | | | | | | | H | E | A | L | | |
| N10K | 1 | N | Q | | V | S | L | T | C | L | V | K | |
| MoA V_{H}CDR₃ | 4 | G | Q | Y | G | N | L | W | F | A | | | Y |

Various publications are cited herein which are hereby incorporated by reference in their entirety.

As will be apparent to those skilled in the art in which the invention is addressed, the present invention may be embodied in forms other than those specifically disclosed above without departing from the spirit or potential characteristics of the invention. Particular embodiments of the present invention described above are therefore to be considered in all respects as illustrative and not restrictive. The scope of the present invention is as set forth in the appended claims and equivalents thereof rather than being limited to the examples contained in the foregoing description.

1. An antimicrobial polypeptide selected from the group consisting of sequences of amino acids as identified by Seq Id Nos. 1, 2, 3, 4, 14, 15, 16, 17, 18, 19, 20, 21, 22, 23, 25, 27, 28, 29, 30, 31, 32, 33, 34, 35, 36, 37, 38, 39, 40, 41, 42, 43, and 44.

2. The antimicrobial peptide of claim 1 wherein the peptide is an antibacterial peptide.

3. The antimicrobial peptide of claim 1 wherein the peptide is an antifungal peptide.

4. The antimicrobial peptide of claim 1 wherein the peptide is an anti-yeast peptide.

5. The antimicrobial peptide of claim 1 wherein the peptide is an anti-mold peptide.

6. An antibacterial peptide selected from the group consisting of sequences of amino acids as identified by Seq Id Nos. 1, 2, 3, 4, 14, 15, 16, 17, 18, 19, 20, 21, 22, and 23.

7. An antifungal peptide selected from the group consisting of sequences of amino acids as identified by Seq Id Nos. 1, 2, 3, 4, 14, 15, 16, 17, 18, 19, 20, 21, 22, 23, 25, 27, 28, 29, 30, 31, 32, 33, 34, 35, 36, 37.

8. An anti-mold peptide selected from the group consisting of sequences of amino acids as identified by Seq Id Nos. 1, 2, 3, 4, 14, 15, 16, 17, 18, 19, 20, 21, 22, 23, 25, 27, 28, 29, 30, 31, 32, 33, 34, 35, 36, 37.

9. An anti-yeast peptide selected from the group consisting of sequences of amino acids as identified by Seq Id Nos. 1, 2, 3, 4, 14, 15, 16, 17, 18, 19, 20, 21, 22, 23, 25, 27, 28, 29, 30, 31, 32, 33, 34, 35, 36, 37.

10. A method of using an antimicrobial peptide in a therapeutic regimen for treating any of a bacterial, fungal, yeast, or mold infection in a mammal comprising administering, either topically or systemically, to said mammal a therapeutically beneficial dosage of said antimicrobial peptide and wherein said peptide is selected from the group consisting of Seq Id Nos. 1, 2, 3, 4, 14, 15, 16, 17, 18, 19, 20, 21, 22, 23, 25, 27, 28, 29, 30, 31, 32, 33, 34, 35, 36, 37.

11. The method of claim 10 wherein the method comprises administration of said peptide topically.

12. The method of claim 10 wherein the method comprises administration of said peptide systemically.

13. The method of claim 10 wherein the peptide is used to treat a bacterial infection.

14. The method of claim 10 wherein the peptide is used to treat a fungal infection.

15. The method of claim 10 wherein the peptide is used to treat a yeast infection.

16. The method of claim 10 wherein the peptide is used to treat a mold infection.

17. A kit comprising a container containing a therapeutic amount of an antimicrobial peptide of claim 1 and a specification and use instructions document.

18. An antiviral peptide selected from the group consisting of sequences of amino acids as identified by Seq Id Nos. 1, 2, and 3.

19. An antitumoral peptide selected from the group consisting of sequences of amino acids as identified by Seq Id Nos. 1, and 2.

20. An immunomodulatory peptide selected from the group consisting of sequences of amino acids as identified by Seq Id Nos. 1, 4, 5, 6, 7, 8, 9, 10, 11, 12, 13, 14, 15, 16, 17, 18, 19, 20, 21, 22, and 23.

21. An anticancer peptide selected from the group consisting of sequences of amino acids as identified by Seq Id Nos. 1, and 2.

22. A method of using an antiviral peptide in a therapeutic regimen for treating a viral infection or disorder in a mammal comprising administering, either topically or systemically, to said mammal a therapeutically beneficial dosage of said antiviral peptide and wherein said peptide is selected from the group consisting of Seq Id Nos. 1, 2, and 3.

23. A method of using an antitumoral peptide in a therapeutic regimen for treating a cancerous disorder in a mammal comprising administering, either topically or systemically, to said mammal a therapeutically beneficial dosage of said antitumoral peptide and wherein said peptide is selected from the group consisting of Seq Id Nos. 1, and 2.

24. A method of using an anticancer peptide in a therapeutic regimen for treating a cancerous disorder in a mammal comprising administering, either topically or systemically, to said mammal a therapeutically beneficial dosage of said anticancer peptide and wherein said peptide is selected from the group consisting of Seq Id Nos. 1, and 2.

25. A method of using an immunomodulatory peptide in a therapeutic regimen for treating any of a microbial, bacterial, fungal, or viral infection, or cancerous or immune disorder in a mammal comprising administering, either topically or systemically, to said mammal a therapeutically beneficial dosage of said immunomodulatory peptide and wherein said peptide is selected from the group consisting of Seq Id Nos. 1, 4, 5, 6, 7, 8, 9, 10, 11, 12, 13, 14, 15, 16, 17, 18, 19, 20, 21, 22, and 23.

26. A polypeptide derived from or having sequence identity with an amino acid sequence of a mammalian immunoglobulin Light (L) or Heavy (H) chain constant domain, said polypeptide of a length of between 3 and 20 amino acids and that exhibits any of antibacterial, antifungal, antiviral, anticancer and immunomodulatory activities.

27. A polypeptide derived from or having sequence identity with an amino acid sequence of a mammalian immunoglobulin Light (L) or Heavy (H) chain constant domain, said polypeptide of a length of between 4 and 11 amino acids and that exhibits any of antibacterial, antifungal, antiviral, anticancer and immunomodulatory activities.

28. A polypeptide derived from or having at least 90% sequence identity with an amino acid sequence of a mammalian immunoglobulin Light (L) or Heavy (H) chain constant domain, said polypeptide of a length of between 4 and 11 amino acids and that exhibits any of antibacterial, antifungal, antiviral, anticancer and immunomodulatory activities.

29. The peptide of any of claims 26, 27 and 28 wherein said peptide is selected from the group consisting of Seq. Id. Nos. 1, 2, 3, 4, 5, 6, 7, 8, 9, 10, 11, 12, 13, 14, 15, 16, 17, 18, 19, 20, 21, 22, 23, 25, 27, 28, 29, 30, 31, 32, 33, 34, 35, 36, 37.

30. The peptide of claim 29 wherein said peptide forms a beta sheet secondary structure.

31. A polypeptide having antimicrobial activities comprising:
between 4 and 11 amino acids said polypeptide having at least between 80 and 90% sequence identity with an Ig L or H chain C region amino acid sequence.

32. The polypeptide of claim 31 wherein said sequence is selected from the group consisting of Seq ID Nos. 1, 2, 3, 4, 14, 15, 16, 17, 18, 19, 20, 21, 22, 23, 24, 25, 27, 28, 29, 30, 31, 32, 33, 34, 35, 36, 37.

33. A polypeptide having antiviral activities comprising:
between 4 and 11 amino acids said polypeptide having at least 90% sequence identity with an Ig L or H chain C region amino acid sequence.

34. The polypeptide of claim 33 wherein said sequence is selected from the group consisting of Seq ID Nos. 1, 2, and 3.

35. A polypeptide having anticancer activities comprising:
between 4 and 11 amino acids said polypeptide having at least 90% sequence identity with an Ig L or H chain C region amino acid sequence.

36. The polypeptide of claim 35 wherein said sequence is selected from the group consisting of Seq ID Nos. 1 and 2.

37. A polypeptide having immunomodulatory activities comprising:
between 4 and 11 amino acids said polypeptide having at least 90% sequence identity with an Ig L or H chain C region amino acid sequence.

38. The polypeptide of claim 37 wherein said sequence is selected from the group consisting of Seq ID Nos. 1, 4, 5, 6, 7, 8, 9, 10, 11, 12, 13, 14, 15, 16, 17, 18, 19, 20, 21, 22, and 23.

39. A bactericidal polypeptide comprising:
a sequence of between 4 and 11 amino acids that in-part exhibits a beta sheet secondary structure, said sequence comprising sequence identity with an IgG or IgM H or L chain C region amino acid sequence.

40. The polypeptide of claim 39 wherein said sequence is selected from Seq Id Nos. 1, 2, 3, 4, 14, 15, 16, 17, 18, 19, 20, 21, 22, and 23.

41. A bactericidal polypeptide comprising:
a sequence of between 4 and 11 amino acids that in-part exhibits a beta sheet secondary structure, said sequence comprising at least 90% sequence identity with an IgG or IgM H or L chain C region amino acid sequence.

42. The polypeptide of claim 41 wherein said sequence is selected from Seq Id Nos. 1, 2, 3, 4, 14, 15, 16, 17, 18, 19, 20, 21, 22, and 23.

43. A method of treating any of a bacterial, fungal, or viral infection of a mammal comprising delivering to said mammal a therapeutically effective amount of a polypeptide selected from the group consisting of polypeptides identified by Seq Id Nos. 1, 2, 3, 4, 5, 6, 7, 8, 9, 10, 11, 12, 13, 14, 15, 16, 17, 18, 19, 20, 21, 22, 23, 25, 27, 28, 29, 30, 31, 32, 33, 34, 35, 36, 37.

44. The method of claim 43 wherein said polypeptide is delivered in an appropriate dosing to achieve an effective concentration selected from the group consisting of a range between 1-2 micrograms/ml, 2-5 micrograms/ml, 3-5 micrograms/ml, 5-10 micrograms/ml, 5-20 micrograms/ml, 30-50 micrograms/ml, 40-50 micrograms/ml, 50-100 micrograms/ml.

45. The method of claim 44 wherein said concentration is selected from the group consisting of 2 micrograms/ml, 5 micrograms/ml, 10 micrograms/ml, 30 micrograms/ml, 50 micrograms/ml, and 100 micrograms/ml.

46. The method of claim 45 wherein said delivery is either by topical or systemic presentation to said mammal and administered either continuously or intermittently to said mammal at dosing frequencies of any of once, twice, three times, or four times in one day, (24 hr period) from one day up to one month.

47. A polypeptide having antimicrobial activity comprising a series of amino acids according to formula X Z*1X 1 Z*11X Z wherein each of X, Z, *, and 1 represent amino acids, wherein Z and * are selected from the group consisting of large charged (basic or acidic), aromatic, Histidine (H), Cysteine (C), Glutamine (Q), Phenylalanine (F), Tryptophan (W), Tyrosine (Y), Lysine (K), Glutamic acid (E), and Arginine (R), wherein X is generally small polar oxygen containing R group selected from the group consisting of Threonine (T), Serine (S), Asparagine (N), and Aspartic acid (D), wherein 1 is hydrophobic nonpolar and generally minimal sized R groups selected from the group consisting of Glycine (G), Valine (V), Leucine (L), Isoleucine (I), Methionine (M), and Alanine (A).

48. A polypeptide of claim 47 wherein said polypeptide is selected from the group consisting of Seq. Id. Nos. 1, 2, 4, 5, 6, 7, 8, 9, 10, 11, 12, 13, 14, 15, 16, 17, 18, 19, 20, 21, 22, 23, 24, 25, 27, 28, 29, 30, 31, 32, 33, 34, 35, 36, and 37.

49. A polypeptide having antimicrobial activity comprising a series of amino acids according to formula Z-B-Hyd-Hyd wherein each of B, Z, and Hyd represent amino acids wherein B is selected from the group consisting of cystine (C), Glutamine (Q), Glutamic acid (E), and Arginine (R), Tyrosine (Y), and Phenylalanine (F), Z is selected from the group consisting of Histidine (H), Cysteine (C), Glutamine (Q), Phenylalanine (F), Tryptophan (W), Tyrosine (Y), Lysine (K), Glutamic acid (E), and Arginine (R), and wherein Hyd is selected from the group consisting of hydrophobic aliphatic and small polar uncharged R group amino acids, Glycine (G), Valine (V) Leucine (L), Isoleucine (I), Methionine (M), and Alanine (A), Serine (S), Threonine (T), Asparagine (N), and Aspartic acid (D).

50. A polypeptide of claim 49 wherein said polypeptide is selected from the group consisting of Seq. Id. Nos. 3, 38, 39, 40, and 41.

51. A polypeptide having antimicrobial activity comprising a series of amino acids according to formula Hyd-X-Z-B-Hyd-Hyd wherein each of B, X, Z, and Hyd represent amino acids wherein B is selected from the group consisting of cystine (C), Glutamine (Q), Glutamic acid (E), and Arginine (R), Tyrosine (Y), and Phenylalanine (F), Z is selected from the group consisting of Histidine (H), Cysteine (C), Glutamine (Q), Phenylalanine (F), Tryptophan (W), Tyrosine (Y), Lysine (K), Glutamic acid (E), and Arginine (R), X is selected from the group consisting of Threonine (T), Serine (S), Asparagine (N), and Aspartic acid (D), and wherein Hyd is selected from the group consisting of hydrophobic aliphatic and small polar uncharged R group amino acids, Glycine (G), Valine (V) Leucine (L), Isoleucine (I), Methionine (M), and Alanine (A), Serine (S), Threonine (T), Asparagine (N), and Aspartic acid (D).

52. A polypeptide of claim 51 wherein said polypeptide is selected from the group consisting of Seq. Id. Nos. 42, 43, and 44.


SEQUENCE LISTING (44 sequences)

SEQ ID NO: 1 - protein, 10 aa, Artificial Sequence
  Description of Artificial Sequence Synthetic peptide
NQVSLTCLVK

SEQ ID NO: 2 - protein, 11 aa, Artificial Sequence
  Description of Artificial Sequence Synthetic peptide
TCRVDHRGLTF

SEQ ID NO: 3 - protein, 4 aa, Artificial Sequence
  Description of Artificial Sequence Synthetic peptide
HEAL

SEQ ID NO: 4 - protein, 10 aa, Artificial Sequence
  Description of Artificial Sequence Synthetic peptide
GQYGNLWFAY

SEQ ID NO: 5 - protein, 10 aa, Artificial Sequence
  Description of Artificial Sequence Synthetic peptide
AQYGNLWFAY

SEQ ID NO: 6 - protein, 10 aa, Artificial Sequence
  Description of Artificial Sequence Synthetic peptide
GAYGNLWFAY

SEQ ID NO: 7 - protein, 10 aa, Artificial Sequence
  Description of Artificial Sequence Synthetic peptide
GQAGNLWFAY

SEQ ID NO: 8 - protein, 10 aa, Artificial Sequence
  Description of Artificial Sequence Synthetic peptide
GQYANLWFAY

SEQ ID NO: 9 - protein, 10 aa, Artificial Sequence
  Description of Artificial Sequence Synthetic peptide
GQYGALWFAY

SEQ ID NO: 10 - protein, 10 aa, Artificial Sequence
  Description of Artificial Sequence Synthetic peptide
GQYGNAWFAY

SEQ ID NO: 11 - protein, 10 aa, Artificial Sequence
  Description of Artificial Sequence Synthetic peptide
GQYGNLAFAY

SEQ ID NO: 12 - protein, 10 aa, Artificial Sequence
  Description of Artificial Sequence Synthetic peptide
GQYGNLWAAY

SEQ ID NO: 13 - protein, 10 aa, Artificial Sequence
  Description of Artificial Sequence Synthetic peptide
GQYGNLWFAA

SEQ ID NO: 14 - protein, 10 aa, Artificial Sequence
  Description of Artificial Sequence Synthetic peptide
AQVSLTCLVK

SEQ ID NO: 15 - protein, 10 aa, Artificial Sequence
  Description of Artificial Sequence Synthetic peptide
NAVSLTCLVK

SEQ ID NO: 16 - protein, 10 aa, Artificial Sequence
  Description of Artificial Sequence Synthetic peptide
NQASLTCLVK

SEQ ID NO: 17 - protein, 10 aa, Artificial Sequence
  Description of Artificial Sequence Synthetic peptide
NQVALTCLVK

SEQ ID NO: 18 - protein, 10 aa, Artificial Sequence
  Description of Artificial Sequence Synthetic peptide
NQVSATCLVK

SEQ ID NO: 19 - protein, 10 aa, Artificial Sequence
  Description of Artificial Sequence Synthetic peptide
NQVSLACLVK

SEQ ID NO: 20 - protein, 10 aa, Artificial Sequence
  Description of Artificial Sequence Synthetic peptide
NQVSLTALVK

SEQ ID NO: 21 - protein, 10 aa, Artificial Sequence
  Description of Artificial Sequence Synthetic peptide
NQVSLTCAVK

SEQ ID NO: 22 - protein, 10 aa, Artificial Sequence
  Description of Artificial Sequence Synthetic peptide
NQVSLTCLAK

SEQ ID NO: 23 - protein, 10 aa, Artificial Sequence
  Description of Artificial Sequence Synthetic peptide
NQVSLTCLVA

SEQ ID NO: 24 - protein, 10 aa, Artificial Sequence
  Description of Artificial Sequence Synthetic peptide
NQVSLTCSVK

SEQ ID NO: 25 - protein, 10 aa, Artificial Sequence
  Description of Artificial Sequence Synthetic peptide
NQVSATCSVK

SEQ ID NO: 26 - protein, 10 aa, Artificial Sequence
  Description of Artificial Sequence Synthetic peptide
MSTAVSKCAT

SEQ ID NO: 27 - protein, 11 aa, Artificial Sequence
  Description of Artificial Sequence Synthetic peptide
ACRVDHRGLTF

SEQ ID NO: 28 - protein, 11 aa, Artificial Sequence
  Description of Artificial Sequence Synthetic peptide
TARVDHRGLTF

SEQ ID NO: 29 - protein, 11 aa, Artificial Sequence
  Description of Artificial Sequence Synthetic peptide
TCAVDHRGLTF

SEQ ID NO: 30 - protein, 11 aa, Artificial Sequence
  Description of Artificial Sequence Synthetic peptide
TCRADHRGLTF

SEQ ID NO: 31 - protein, 11 aa, Artificial Sequence
  Description of Artificial Sequence Synthetic peptide
TCRVAHRGLTF

SEQ ID NO: 32 - protein, 11 aa, Artificial Sequence
  Description of Artificial Sequence Synthetic peptide
TCRVDARGLTF

SEQ ID NO: 33 - protein, 11 aa, Artificial Sequence
  Description of Artificial Sequence Synthetic peptide
TCRVDHAGLTF

SEQ ID NO: 34 - protein, 11 aa, Artificial Sequence
  Description of Artificial Sequence Synthetic peptide
TCRVDHRALTF

SEQ ID NO: 35 - protein, 11 aa, Artificial Sequence
  Description of Artificial Sequence Synthetic peptide
TCRVDHRGATF

SEQ ID NO: 36 - protein, 11 aa, Artificial Sequence
  Description of Artificial Sequence Synthetic peptide
TCRVDHRGLAF

SEQ ID NO: 37 - protein, 11 aa, Artificial Sequence
  Description of Artificial Sequence Synthetic peptide
TCRVDHRGLTA

SEQ ID NO: 38 - protein, 4 aa, Artificial Sequence
  Description of Artificial Sequence Synthetic peptide
CRVD

SEQ ID NO: 39 - protein, 4 aa, Artificial Sequence
  Description of Artificial Sequence Synthetic peptide
HRGL

SEQ ID NO: 40 - protein, 4 aa, Artificial Sequence
  Description of Artificial Sequence Synthetic peptide
TCLV

SEQ ID NO: 41 - protein, 4 aa, Artificial Sequence
  Description of Artificial Sequence Synthetic peptide
QYGN

SEQ ID NO: 42 - protein, 6 aa, Artificial Sequence
  Description of Artificial Sequence Synthetic peptide
VDHRGL

SEQ ID NO: 43 - protein, 6 aa, Artificial Sequence
  Description of Artificial Sequence Synthetic peptide
VSLTCL

SEQ ID NO: 44 - protein, 6 aa, Artificial Sequence
  Description of Artificial Sequence Synthetic peptide
GNLWFA